SEQUENCE LISTING (22 sequences)

SEQ ID NO: 1 - protein, 10 aa, Homo sapiens
RVWDVSGLRK

SEQ ID NO: 2 - protein, 11 aa, Homo sapiens
RVWDVSGLRKK

SEQ ID NO: 3 - protein, 9 aa, Homo sapiens
LLDGFLATV

SEQ ID NO: 4 - protein, 10 aa, Homo sapiens
SLLDGFLATV

SEQ ID NO: 5 - protein, 11 aa, Homo sapiens
AENALESYAFN

SEQ ID NO: 6 - protein, 10 aa, Homo sapiens
AENAEFMRNF

SEQ ID NO: 7 - protein, 11 aa, Homo sapiens
GLADGVMQCSF

SEQ ID NO: 8 - protein, 9 aa, Homo sapiens
AVADDFCKV

SEQ ID NO: 9 - protein, 9 aa, Homo sapiens
SPRQPPLLL

SEQ ID NO: 10 - protein, 9 aa, Homo sapiens
LLDRFLATV

SEQ ID NO: 11 - protein, 10 aa, Homo sapiens
SLLDRFLATV

SEQ ID NO: 12 - protein, 10 aa, Homo sapiens
RVWDISGLRK

SEQ ID NO: 13 - protein, 11 aa, Homo sapiens
RVWDISGLRKK

SEQ ID NO: 14 - protein, 9 aa, Homo sapiens
SPQQPPLLL

SEQ ID NO: 15 - DNA, 21 bp, Artificial Sequence
  Synthetic
gatgtggaaagtgaatgtgcg

SEQ ID NO: 16 - DNA, 22 bp, Artificial Sequence
  Synthetic
tttggatgagcctttacggtag

SEQ ID NO: 17 - DNA, 21 bp, Artificial Sequence
  Synthetic
cactagggaagcacagatgtg

SEQ ID NO: 18 - DNA, 22 bp, Artificial Sequence
  Synthetic
ccaatggtgtggctgtgtgaag

SEQ ID NO: 19 - DNA, 21 bp, Artificial Sequence
  Synthetic
gacaccgrcactgccaccttc

SEQ ID NO: 20 - DNA, 20 bp, Artificial Sequence
  Synthetic
ggtagatactcagttcctgg

SEQ ID NO: 21 - DNA, 19 bp, Artificial Sequence
  Synthetic
catcatctctgccccctct

SEQ ID NO: 22 - DNA, 20 bp, Artificial Sequence
  Synthetic
ggtgctaagcagttggtggt

(19) United States
(12) Patent Application Publication
(10) Pub. No.: US 2021/0172961 A1
(43) Pub. Date: Jun. 10, 2021

(54) METHODS FOR IDENTIFYING RNA EDITING-DERIVED EPITOPES THAT ELICIT IMMUNE RESPONSES IN CANCER

(71) Applicants: Board of Regents, The University of Texas System, Austin, TX (US); Immatics Biotechnologies GmbH, Tuebingen (DE)

(72) Inventors: Minying ZHANG, Houston, TX (US); Jens FRITSCHE, Dusslingen (DE); Patrick HWU, Houston, TX (US)

(21) Appl. No.: 17/263,094

(22) PCT Filed: Jul. 26, 2019
(86) PCT No.: PCT/US19/43606

§ 371 Date: Jan. 25, 2021

Related U.S. Application Data
(60) Provisional application No. 62/711,175, filed on Jul. 27, 2018.

(51) Int. Cl. G01N 33/68; G01N 33/50

(52) U.S. Cl. CPC G01N 33/6878; G01N 33/505; G01N 2333/70539; G01N 33/6848; G01N 33/6818

(57) ABSTRACT

The present disclosure relates to methods of identifying RNA-edited peptides. In an aspect, the identified peptides are capable of eliciting immune responses in individuals or patients. The present disclosure further relates to RNA-edited peptide sequences identified by methods described herein. In a further aspect, the disclosure provides for methods of treating cancer in individuals or patients by utilizing the methodology described herein.

### CROSS REFERENCE TO RELATED APPLICATIONS

This application claims the benefit of U.S. Provisional Application Ser. No. 62/711,175, filed Jul. 27, 2018. The content of this application is herein incorporated by reference in their entirety.

### STATEMENT REGARDING FEDERALLY SPONSORED RESEARCH OR DEVELOPMENT

This invention was made with government support under grant numbers CA016672, CA175486, and CA168394 awarded by the National Institutes of Health. The government has certain rights in the invention.

### REFERENCE TO SEQUENCE LISTING SUBMITTED AS A COMPLIANT ASCII TEXT FILE (.TXT)

Pursuant to the EFS-Web legal framework and 37 CFR §§ 1.821-825 (see MPEP § 2442.03(a)), a Sequence Listing in the form of an ASCII-compliant text file (entitled “Sequence_Listing_3000018-003977_ST25.txt” created on 26 Jul. 2019, and 3,698 bytes in size) is submitted concurrently with the instant application, and the entire contents of the Sequence Listing are incorporated herein by reference.

### BACKGROUND

### Field

The present disclosure relates to methods of identifying RNA-edited peptides. In an aspect, the identified peptides are capable of eliciting immune responses in individuals or patients. The present disclosure further relates to RNA-edited peptide sequences identified by methods described herein. In a further aspect, the disclosure provides for methods of treating cancer in individuals or patients by utilizing the methodology described herein.

### Background

T-cell based immunotherapy targets peptide epitopes derived from tumor-associated or tumor-specific proteins, which are presented by molecules of the major histocompatibility complex (MHC). The antigens that are recognized by the tumor specific T lymphocytes, that is, the epitopes thereof, can be molecules derived from all protein classes, such as enzymes, receptors, transcription factors, etc. which are expressed and, as compared to unaltered cells of the same origin, usually up-regulated in cells of the respective tumor.

RNA editing is a post-transcriptional processing mechanism that results in an RNA sequence that is different from that encoded by the genomic DNA and thereby diversifies the gene product and function. The type of RNA editing that is most prevalent in higher eukaryotes converts adenosine residues into inosine (A-to-I editing) in double-stranded RNA (dsRNA) through the action of double-stranded RNA-specific adenosine deaminases. During translation, inosine is read as guanosine, and therefore this mechanism can change codons in mRNAs. These changes can affect protein structure and function. Any codon change, which requires the conversion of adenosine to guanosine, is possible. RNA editing by adenosine deamination is believed to occur in most metazoans. RNA-specific adenosine deaminases include ADAR1 (also known as ADAR), which destabilizes double-stranded RNA through conversion of adenosine to inosine. The ADAR1 enzyme modifies cellular and viral RNAs, including coding and noncoding RNAs. ADAR1 targets double-stranded RNA hairpin-containing loop structures, such as microRNAs (miRNAs) by operating through base-pairing with complementary sequences within an mRNA molecule leading to mRNA degradation and gene silencing. ADAR activity is suggested in various tumor types.

A-to-I RNA editing may contribute to cancer development and progression. For example, ADAR1 was downregulated when growth rates of HeLa-cell-derived tumors in xenograft model were inhibited. ADAR1 deletion leads to regression of established chronic myelogenous leukemia in mice. In addition, some cancer-related RNA editing targets were discovered, such as antizyme inhibitor 1 (AZIN1) and glioma-associated oncogene 1 (GLI1). A-to-I RNA editing of AZIN1 is increased in hepatocellular carcinoma. RNA editing of GLI1 transcription factor involved in Hedgehog signaling is decreased in basal cell carcinoma tumor

While U.S. Patent Application No. 2017/0191057 describes a method of measuring and tracing the A-to-I RNA editing changes in cancer stem cells, this application does not disclose edited MHC peptides presented on the surface of cancer cells due to the A-to-I RNA editing changes.

There remains a need for identifying MHC peptides as suitable targets for immunotherapy. The solution to this technical problem is provided by the embodiments characterized in the claims.

### BRIEF SUMMARY

In an aspect, the present disclosure relates to methods for identifying an RNA editing-derived epitope that elicits an immune response in an individual, patient, or subject.

In an aspect, methods for identifying an RNA editing-derived epitope described herein include, for example:
(a) isolating a plurality of MHC epitopes from an individual, patient, or subject;
(b) selecting, from the plurality of MHC epitopes, an MHC epitope comprising an edited amino acid sequence,
(c) activating an MHC epitope-specific T cell,
(d) isolating the activated MHC epitope-specific T cell,
(e) contacting the isolated activated MHC epitope-specific T cell with a target cell, and
(f) identifying the selected MHC epitope as the RNA editing-derived epitope.

In another aspect, methods for identifying an RNA editing-derived epitope described herein include, for example:
(a) isolating a plurality of MHC epitopes from an individual, patient, or subject;
(b) selecting, from the plurality of MHC epitopes, an MHC epitope comprising an edited amino acid sequence,
(c) activating an MHC epitope-specific T cell in peripheral blood mononuclear cells (PBMC) by contacting the PBMC with an antigen presenting cell presenting the selected MHC epitope on the cell surface,
(d) isolating the activated MHC epitope-specific T cell from the PBMC,
(e) contacting the isolated activated MHC epitope-specific T cell with a target cell, and
(f) identifying the selected MHC epitope as the RNA editing-derived epitope.

In an aspect, the MHC epitope is identified as an RNA editing-derived epitope if contacting the isolated activated MHC epitope-specific T cell with the target cell elicits an immune response against the target cell.

In another aspect, the edited amino acid sequence is obtained from an RNA editome peptide database containing an RNA editing site and the corresponding amino acid sequence.

In another aspect, methods described herein further include performing a spectrometry analysis, for example, a mass spectrometer analysis, on the plurality of MHC epitopes to generate a high-resolution spectrum and a low-resolution spectrum for each of the plurality of MHC epitopes. In another aspect, the mass spectrometer parameters include a mass range about 700-1500 Da, a precursor mass tolerance about 3 ppm, about 0.02 Da bin size for the high-resolution spectra, and about 1 Da for the low-resolution spectra.

In another aspect, the selected MHC epitope has a length of from 8 to 12 amino acids.

In another aspect, the individual, patient, or subject has cancer. In an aspect, the individual, patient, or subject is a human.

In another aspect, the cancer is selected from the group consisting of glioblastoma, breast cancer, colorectal cancer, renal cell carcinoma, chronic lymphocytic leukemia, hepatocellular carcinoma, non-small cell and small cell lung cancer, Non-Hodgkin lymphoma, acute myeloid leukemia, ovarian cancer, pancreatic cancer, prostate cancer, esophageal cancer including cancer of the gastric-esophageal junction, gallbladder cancer and cholangiocarcinoma, melanoma, gastric cancer, testis cancer, urinary bladder cancer, head and neck squamous cell carcinoma, and uterine cancer.

In another aspect, the antigen presenting cell is a dendritic cell.

In another aspect, the immune response includes a cytotoxic T cell response.

In another aspect, the immune response includes IFN-γ release by the isolated activated MHC epitope-specific T cell.

In another aspect, the immune response is capable of killing the target cell.

In another aspect, the RNA editing-derived epitope comprises, consists of, or consists essentially of a peptide with amino acid sequence selected from the group consisting of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9).

In an aspect, the peptide is in the form of a pharmaceutically acceptable salt.

In another aspect, the individual, patient, or subject expresses an RNA-specific adenosine deaminase (ADAR) gene.

In another aspect, the ADAR gene is ADAR1 gene. In another aspect, the ADAR converts adenosine to inosine.

In another aspect, the target cell presents the RNA editing-derived epitope in a complex with an MHC molecule on the cell surface.

In an aspect, peptides identified from the methodology described herein are used in methods of eliciting an immune response in a patient, subject, or individual who has cancer.

In another aspect, the disclosure provides for methods of eliciting an immune response in a patient, subject, or individual who has cancer, comprising administering to the patient, subject, or individual a population of activated T cells that selectively recognize cancer cells that present a peptide on the cell surface, wherein the peptide comprises, consists essentially of, or consists of an amino acid sequence selected from the group consisting of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9).

In an aspect, the cancer is selected from the group consisting of glioblastoma, breast cancer, colorectal cancer, renal cell carcinoma, chronic lymphocytic leukemia, hepatocellular carcinoma, non-small cell and small cell lung cancer, Non-Hodgkin lymphoma, acute myeloid leukemia, ovarian cancer, pancreatic cancer, prostate cancer, esophageal cancer including cancer of the gastric-esophageal junction, gallbladder cancer and cholangiocarcinoma, melanoma, gastric cancer, testis cancer, urinary bladder cancer, head and neck squamous cell carcinoma, and uterine cancer.

In another aspect, the activated T cells described herein are produced by contacting T cells with the peptide loaded human class I or II MHC molecules expressed on the surface of an antigen-presenting cell for a period of time sufficient to activate the T cells.

In an aspect, the activated T cells are expanded in vitro. In another aspect, the contacting is in vitro.

In another aspect, the T cells are autologous to the patient.

In yet another aspect, the T cells are obtained from a healthy donor.

In an aspect, the T cells are obtained from tumor infiltrating lymphocytes or peripheral blood mononuclear cells.

In another aspect, the peptide is in a complex with the class I MHC molecule.

In yet another aspect, the antigen presenting cell is infected with recombinant virus expressing the peptide.

In an aspect, the antigen presenting cell is a dendritic cell or a macrophage.

In another aspect, the expansion is in the presence of an anti-CD28 antibody and IL-12.

In yet another aspect, the population of activated T cells includes CD8-positive cells.

In an aspect, the population of activated T cells are administered in the form of a composition. In another aspect, the composition includes an adjuvant. In yet another aspect, the adjuvant is selected from the group consisting of anti-CD40 antibody, imiquimod, resiquimod, GM-CSF, cyclophosphamide, sunitinib, bevacizumab, interferon-alpha, interferon-beta, CpG oligonucleotides and derivates, poly-(I:C) and derivates, RNA, sildenafil, particulate formulations with poly(lactid co-glycolid) (PLG), virosomes, interleukin (IL)-1, IL-2, IL-4, IL-7, IL-12, IL-13, IL-15, IL-21, and IL-23.

In an aspect, the immune response is capable of killing the cancer cells. In another aspect, the immune response includes a cytotoxic T cell response. In yet another aspect, the immune response includes IFN-γ release by the activated T cell.

In another aspect, the cancer cells express an RNA-specific adenosine deaminase (ADAR) gene. In another aspect, the ADAR gene is ADAR1 gene.

In an aspect, the present disclosure relates to methods of treating a patient, subject, or individual who has cancer, including administering to the patient a population of activated T cells that selectively recognize cancer cells that present a peptide on the cell surface, wherein the peptide comprises, consists essentially of, or consists of an amino acid sequence selected from the group consisting of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9).

In another aspect, the activated T cells are produced by contacting T cells with the peptide loaded human class I or II MHC molecules expressed on the surface of an antigen-presenting cell for a period of time sufficient to activate the T cells.

In yet another aspect, the cancer is selected from the group consisting of glioblastoma, breast cancer, colorectal cancer, renal cell carcinoma, chronic lymphocytic leukemia, hepatocellular carcinoma, non-small cell and small cell lung cancer, Non-Hodgkin lymphoma, acute myeloid leukemia, ovarian cancer, pancreatic cancer, prostate cancer, esophageal cancer including cancer of the gastric-esophageal junction, gallbladder cancer and cholangiocarcinoma, melanoma, gastric cancer, testis cancer, urinary bladder cancer, head and neck squamous cell carcinoma, and uterine cancer.

In an aspect, the T cells are autologous to the patient. In another aspect, the T cells are obtained from a healthy donor. In yet another aspect, the T cells are obtained from tumor infiltrating lymphocytes or peripheral blood mononuclear cells.

In an aspect, the activated T cells are expanded in vitro. In another aspect, the contacting is in vitro.

In an aspect, the peptide is in a complex with the class I MHC molecule.

In another aspect, the antigen presenting cell is infected with recombinant virus expressing the peptide.

In an aspect, the antigen presenting cell is a dendritic cell or a macrophage. In an aspect, the expansion is in the presence of an anti-CD28 antibody and IL-12. In a further aspect, the population of activated T cells includes CD8-positive cells. In yet another aspect, the population of activated T cells are administered in the form of a composition.

In another aspect, the activated T cells are capable of killing the cancer cells.

In another aspect, the activated T cells include cytotoxic T cells.

In another aspect, the activated T cells contacting the cancer cells are capable of releasing IFN-γ.

In another aspect, the cancer cells express an RNA-specific adenosine deaminase (ADAR) gene.

In another aspect, the ADAR gene is ADAR1 gene.

In an aspect, the disclosure provides for methods comprising, consisting essentially of, or consisting of any of the method steps described herein.

In another aspect, the present disclosure relates to an antibody that specifically binds a peptide consisting of the amino acid sequence selected from the group consisting of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9) or a human major histocompatibility complex (MHC) complexed with the peptide.

In another aspect, the antibody is a polyclonal antibody, monoclonal antibody, bi-specific antibody, or a chimeric antibody.

In another aspect, the MHC complexed with the peptide is present in a tumor cell.

In another aspect, the tumor cell is at least one selected from the group consisting of glioblastoma, breast cancer, colorectal cancer, renal cell carcinoma, chronic lymphocytic leukemia, hepatocellular carcinoma, non-small cell and small cell lung cancer, Non-Hodgkin lymphoma, acute myeloid leukemia, ovarian cancer, pancreatic cancer, prostate cancer, esophageal cancer including cancer of the gastric-esophageal junction, gallbladder cancer and cholangiocarcinoma, melanoma, gastric cancer, testis cancer, urinary bladder cancer, head and neck squamous cell carcinoma, and uterine cancer.

In another aspect, the antibody is labeled with a toxin.

In another aspect, the antibody is labeled with a radionucleotide.

In another aspect, the radionucleotide is ¹¹¹In, ⁹⁹Tc, ¹⁴C, ¹³¹I, ³H, ³²P or ³⁵S.

In another aspect, the affinity value (Kd) of the antibody is less than 1×10 μM.

In another aspect, the MHC is a MHC class I molecule.

In another aspect, the MHC is a MHC class II molecule.

In another aspect, the antibody further comprises a binding affinity of below 20 nanomolar.

In another aspect, the antibody is humanized.

In another aspect, the present disclosure relates to a T-cell receptor (TCR) optionally a soluble or membrane-bound TCR or functional fragment thereof that is reactive with a peptide consisting of the amino acid sequence selected from the group consisting of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9) in a complex with an MHC molecule.

In another aspect, the TCR is provided as a soluble molecule and optionally carries a further effector function optionally an antibody fragment, an immune stimulating domain and/or a toxin.

In another aspect, the TCR is an alpha/beta heterodimeric TCR comprising alpha and beta chain constant domain sequences, in which cysteine residues are substituted for Thr 48 of TCR a constant region (TRAC) and Ser 57 of TCR 13 constant region 1 (TRBC1) or TCR 13 constant region 2 (TRBC2) and form a disulfide bond between the alpha and beta constant domains of the TCR.

In an aspect, the substitution is a conservative amino acid substitution. In an aspect, the amino acid substitutions may be located within a CDR of the TCR, e.g., one or more of CDR1, CDR2, and CDR3. In an aspect, the amino acid substations may be located within the CDR1α, CDR2α, CDR3α, CDR1β, CDR2β, and/or CDR3β chain of the TCR. In an aspect, an amino acid substitution, for example, a conservative substitution, is the first or last amino acid residue of the respective CDR sequence.

In an aspect, the disclosure provides for CDR1, CDR2, and CDR3 variants having modified amino acid residues. A modified amino acid residue may be selected from an amino acid insertion, deletion, or substitution.

In an aspect, a substitution described herein is a conservative amino acid substitution. That is, amino acids of CDRs may be replaced by other amino acids having similar properties (conservative changes, such as similar hydrophobicity, hydrophilicity, antigenicity, propensity to form or break a-helical structures or 3-sheet structures). Nonlimiting examples of conservative substitutions may be found in, for example, Creighton (1984) Proteins. W. H. Freeman and Company, the contents of which are incorporated by reference in their entirety.

In an aspect, conservative substitutions may include those, which are described by Dayhoff in “The Atlas of Protein Sequence and Structure. Vol. 5”, Natl. Biomedical Research, the contents of which are incorporated by reference in their entirety. For example, in an aspect, amino acids, which belong to one of the following groups, can be exchanged for one another, thus, constituting a conservative exchange: Group 1: alanine (A), proline (P), glycine (G), asparagine (N), serine (S), threonine (T); Group 2: cysteine (C), serine (S), tyrosine (Y), threonine (T); Group 3: valine (V), isoleucine (I), leucine (L), methionine (M), alanine (A), phenyl-alanine (F); Group 4: lysine (K), arginine (R), histidine (H); Group 5: phenylalanine (F), tyrosine (Y), tryptophan (W), histidine (H); and Group 6: aspartic acid (D), glutamic acid (E). In an aspect, a conservative amino acid substitution may be selected from the following of T→A, G→A, A→I, T→V, A→M, T→I, A→V, T→G, and/or T→S.

In an aspect, a conservative amino acid substitution may include the substitution of an amino acid by another amino acid of the same class, for example, (1) nonpolar: Ala, Val, Leu, Ile, Pro, Met, Phe, Trp; (2) uncharged polar: Gly, Ser, Thr, Cys, Tyr, Asn, Gln; (3) acidic: Asp, Glu; and (4) basic: Lys, Arg, His. Other conservative amino acid substitutions may also be made as follows: (1) aromatic: Phe, Tyr, His; (2) proton donor: Asn, Gln, Lys, Arg, His, Trp; and (3) proton acceptor: Glu, Asp, Thr, Ser, Tyr, Asn, Gln (see, for example, U.S. Pat. No. 10,106,805, the contents of which are incorporated by reference in their entirety).

In another aspect, conservative substitutions may be made in accordance with Table A. Methods for predicting tolerance to protein modification may be found in, for example, Guo et al., Proc. Natl. Acad. Sci., USA, 101(25):9205-9210 (2004), the contents of which are incorporated by reference in their entirety.

In an aspect, a substitution described herein comprises an amino acid not naturally present in a region of the antigen recognizing construct. In another aspect, a substitution modification described herein comprises an amino acid not naturally present in a region of the respective CDR region, for example any of CDR1, CDR2, or CDR3 of an antigen recognizing construct described herein.

In another aspect, the TCR is an alpha/beta heterodimeric TCR comprising alpha and beta chain constant domain sequences, in which the constant domain sequences are linked by a native disulfide bond between Cys4 of exon 2 either of TRAC and Cys2 of exon 2 of either TRBC1 or TRBC2.

In another aspect, the TCR is associated with a detectable label, a therapeutic agent, a PK modifying moiety or any combination thereof.

In another aspect, the therapeutic agent is an anti-CD3 antibody covalently linked to the C- or N-terminus of the alpha or beta chain of the TCR.

In another aspect, a nucleic acid encoding for the TCR is optionally linked to a heterologous promoter sequence, or an expression vector capable of expressing said nucleic acid.

In another aspect, a host cell comprising the nucleic acid or an expression vector capable of expressing the nucleic acid, in which the host cell optionally is a T cell or NK cell.

In another aspect, the present disclosure relates to an aptamer that specifically binds to a peptide consisting of the amino acid sequence selected from the group consisting of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9) or a human major histocompatibility complex (MHC) complexed with the peptide.

In another aspect, the present disclosure relates to a pharmaceutical composition comprising a peptide consisting of the amino acid sequence selected from the group consisting of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9) in the form of a pharmaceutically acceptable salt and an adjuvant.

In another aspect, the salt is a chloride salt or an acetate salt.

In another aspect, the adjuvant is selected from the group consisting of anti-CD40 antibody, imiquimod, resiquimod, GM-CSF, cyclophosphamide, sunitinib, bevacizumab, interferon-alpha, interferon-beta, CpG oligonucleotides and derivates, poly-(I:C) and derivates, RNA, sildenafil, particulate formulations with poly(lactid co-glycolid) (PLG), virosomes, interleukin (IL)-1, IL-2, IL-4, IL-7, IL-12, IL-13, IL-15, IL-21, and IL-23.

### BRIEF DESCRIPTION OF THE DRAWINGS

FIG. 1 shows proteogenomics-guided discovery of HLA peptides derived from RNA editing in accordance with an embodiment of the present disclosure.

FIGS. 2A-2L show LC-MS/MS validation of peptide sequence identity based on identical MS/MS spectrum and coelution of synthetic reference in accordance with one embodiment of the present disclosure.

FIGS. 3A and 3B show relative abundance of HLA-bound peptides derived from non-edited wild type (WT) and edited (ED) peptides isolated from tumour and normal samples in accordance with one embodiment of the present disclosure.

FIGS. 4A-4D show response of T cells to edited peptides in accordance with one embodiment of the present disclosure.

FIGS. 5A and 5B show confirmation of wildtype and edited gene and protein expression in accordance with one embodiment of the present disclosure.

FIGS. 6A-6F show correlation of edited peptide levels with gene and ADAR mRNA expression as well as Ted10 activation and Ted10 mediated tumor target killing in accordance with one embodiment of the present disclosure.

FIGS. 7A-7B show TCGA data analysis in accordance with one embodiment of the present disclosure.

FIGS. 8A-8C show knockdown of ADAR1 in accordance with one embodiment of the present disclosure.

### DETAILED DESCRIPTION

In addition to genomic mutations, RNA editing is a mechanism for creating point variations in proteins by introducing nucleotide changes in RNA sequences. The most common RNA editing, which converts adenosine to inosine (A→I editing), may be catalyzed by a family of adenosine deaminases (ADARs). Deregulated RNA editing and the resulting protein alterations may contribute to different types of human diseases, including cancers. As described herein, peptides derived from RNA edited transcripts and presented on human leukocyte antigen (HLA) may be capable of serving as a source for cancer antigens.

In an aspect, the disclosure provides for methods of employing RNA editing to identify antigens, for example, cancer antigens. In another aspect, the present disclosure relates to identification of RNA editing-derived epitopes that an elicit immune response in an individual or subject. The present disclosure also relates to identification of RNA editing-derived epitopes by selecting MHC epitopes that contain edited amino acid sequences. In another aspect, the present disclosure relates to identification of RNA editing-derived epitopes by contacting the isolated activated MHC epitope-specific T cell with a target cell and identifying the selected MHC epitope as the RNA editing-derived epitope.

In a further aspect, the present disclosure relates to identification of RNA editing-derived epitopes that elicit immune response by analyzing HLA ligandome from normal samples, different organs, cancer samples from different cancer indications, proteogenomics screening of HLA bound peptides using LC-MS and/or MS/MS and RNA sequencing RNA editing sites, and characterizing candidate RNA editing-derived peptides on HLA peptide levels (quantification map of peptide presentation), T cell levels (activation killing), and mRNA levels (correlation analysis).

In an aspect, peptides generated as a consequence of RNA editing are presented by HLA and are capable of functioning as tumour antigens to elicit immune responses. In another aspect, effector CD8+ T cells specific for edited peptides are present in human tumours and attack tumour cells that are presenting these epitopes. In addition, quantification of the edited peptides shows that the prevalence of cancer patients with hyper-edited levels of HLA-bound peptide may be comparable to the most frequently shared neoantigens and that edited RNA can predict absolute peptide copy numbers.

In yet another aspect, the present disclosure relates to methods of eliciting an immune response in a patient, subject, or individual who has cancer. The present disclosure also relates to eliciting an immune response in a patient, subject, or individual who has cancer by administering to the patient a population of activated T cells that selectively recognize cancer cells that present peptide described herein on the cell surface.

The present disclosure further relates to eliciting an immune response in a patient, subject, or individual who has cancer. Methods of treating a patient, subject, or individual who has cancer are also provided herein. The present disclosure relates to the RNA editing-derived peptides according to the present disclosure for use in the treatment of proliferative diseases.

In an aspect, the cancer to be treated is selected from the group consisting of, for example, glioblastoma (GB), breast cancer (BRCA), colorectal cancer (CRC), renal cell carcinoma (RCC), chronic lymphocytic leukemia (CLL), hepatocellular carcinoma (HCC), non-small cell and small cell lung cancer (NSCLC, SCLC), Non-Hodgkin lymphoma (NHL), acute myeloid leukemia (AML), ovarian cancer (OC), pancreatic cancer (PC), prostate cancer (PCA), esophageal cancer including cancer of the gastric-esophageal junction (OSCAR), gallbladder cancer and cholangiocarcinoma (GBC, CCC), melanoma (MEL), gastric cancer (GC), urinary bladder cancer (UBC), head- and neck squamous cell carcinoma (HNSCC), uterine cancer (UEC), and any cancers that express ADAR proteins, e.g., ADAR1.

The present disclosure further relates to a method of killing target cells in an individual which target cells aberrantly express a polypeptide comprising any amino acid sequence according to the present disclosure, the method comprising administering to the individual an effective number of T cells as produced according to the present disclosure.

In another aspect, the present disclosure relates to RNA editing-derived peptides according to the present disclosure that have the ability to bind to a molecule of the human major histocompatibility complex (MHC) class-I or—in an elongated form, such as a length-variant—MHC class-II.

The present disclosure further relates to RNA editing-derived peptides comprising, consisting of, or consisting essentially one or more of the amino acid sequences selected from the group consisting of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9).

In an aspect, RNA editing-derived peptides described herein or identified by methods described herein may be further modified and/or include non-peptide bonds.

In another aspect, RNA editing-derived peptides described herein or identified by methods described herein are part of a fusion protein, for example, fused to the N-terminal amino acids of the HLA-DR antigen-associated invariant chain (Ii), or fused to (or into the sequence of) an antibody, such as, for example, an antibody that is specific for dendritic cells.

In another aspect, the present disclosure relates to antibodies capable of binding to RNA editing-derived peptides described herein, such as RNA editing-derived peptides comprising, consisting of, or consisting essentially one or more of the amino acid sequences selected from the group consisting of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9). The antibodies may be monoclonal antibodies, polyclonal antibodies, and humanized antibodies.

The term “monoclonal antibody” as used herein refers to an antibody obtained from a substantially homogeneous population of antibodies, i.e.; the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired antagonistic activity (U.S. Pat. No. 4,816,567, which is hereby incorporated in its entirety).

Monoclonal antibodies of the present disclosure may be prepared using hybridoma methods. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods, such as those described in U.S. Pat. No. 4,816,567, the content of which is incorporated by reference in its entirety. DNA encoding the monoclonal antibodies of the invention can be readily isolated and sequenced using conventional procedures (e.g., by using oligonucleotide probes that are capable of binding specifically to genes encoding the heavy and light chains of murine antibodies).

In vitro methods are also suitable for preparing monovalent antibodies. Digestion of antibodies to produce fragments thereof, particularly Fab fragments, can be accomplished using routine techniques known in the art. For instance, digestion can be performed using papain. Examples of papain digestion are described in WO 94/29348 and U.S. Pat. No. 4,342,566, the contents of which are incorporated by reference in their entireties. Papain digestion of antibodies typically produces two identical antigen binding fragments, called Fab fragments, each with a single antigen binding site, and a residual Fc fragment. Pepsin treatment yields a F(ab′)2 fragment and a pFc′ fragment.

The antibody fragments, whether attached to other sequences or not, can also include insertions, deletions, substitutions, or other selected modifications of particular regions or specific amino acids residues, provided the activity of the fragment is not significantly altered or impaired compared to the non-modified antibody or antibody fragment. These modifications can provide for additional properties, such as increasing bio-longevity or altering secretory characteristics.

Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody fragment.

The antibodies of the present disclosure may further comprise humanized antibodies or human antibodies. Humanized forms of non-human (e.g., murine) antibodies are chimeric immunoglobulins, immunoglobulin chains or fragments thereof (such as Fv, Fab, Fab′ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity. In some instances, Fv framework (FR) residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fe), typically that of a human immunoglobulin.

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region gene in chimeric and germ-line mutant mice results in complete inhibition of endogenous antibody production. Transfer of the human germ-line immunoglobulin gene array in such germ-line mutant mice will result in the production of human antibodies upon antigen challenge. Human antibodies can also be produced in phage display libraries.

Antibodies of the present disclosure may be administered to a subject in a pharmaceutically acceptable carrier. Typically, an appropriate amount of a pharmaceutically-acceptable salt is used in the formulation to render the formulation isotonic. Examples of the pharmaceutically-acceptable carrier include saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of antibody being administered.

The antibodies can be administered to the subject, patient, or cell by injection (e.g., intravenous, intraperitoneal, subcutaneous, intramuscular), or by other methods such as infusion that ensure its delivery to the bloodstream in an effective form. The antibodies may also be administered by intratumoral or peritumoral routes, to exert local as well as systemic therapeutic effects. Local or intravenous injection is preferred.

The present disclosure further relates to T-cell receptors (TCRs) that bind to a MHC molecule/RNA editing-derived peptide complex. RNA editing-derived peptides described herein comprise, consist of, or consist essentially of one or more of the amino acid sequences selected from the group consisting of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9).

In an aspect, TCRs described herein comprise an alpha chain and a beta chain (“alpha/beta TCRs”). Also provided are peptides capable of binding to TCRs and antibodies when presented by an MHC molecule. The present disclosure also relates to nucleic acids, vectors and host cells for expressing TCRs and peptides of the present description; and methods of using the same.

The term “T-cell receptor” (abbreviated TCR) refers to a heterodimeric molecule comprising an alpha polypeptide chain (alpha chain) and a beta polypeptide chain (beta chain), wherein the heterodimeric receptor is capable of binding to a peptide antigen presented by an HLA molecule. The term also includes so-called gamma/delta TCRs.

In one embodiment, the present disclosure provides a method of producing a TCR as described herein, the method comprising culturing a host cell capable of expressing the TCR under conditions suitable to promote expression of the TCR.

In another aspect relates to methods according to the present disclosure, wherein the antigen is loaded onto class I or II MHC molecules expressed on the surface of a suitable antigen-presenting cell or artificial antigen-presenting cell by contacting a sufficient amount of the antigen with an antigen-presenting cell or the antigen is loaded onto class I or II MHC tetramers by tetramerizing the antigen/class I or II MHC complex monomers.

The alpha and beta chains of alpha/beta TCR's, and the gamma and delta chains of gamma/delta TCRs, are generally regarded as each having two “domains”, namely variable and constant domains. The variable domain consists of a concatenation of variable region (V), and joining region (J). The variable domain may also include a leader region (L). Beta and delta chains may also include a diversity region (D). The alpha and beta constant domains may also include C-terminal transmembrane (TM) domains that anchor the alpha and beta chains to the cell membrane.

With respect to gamma/delta TCRs, the term “TCR gamma variable domain” as used herein refers to the concatenation of the TCR gamma V (TRGV) region without leader region (L), and the TCR gamma J (TRGJ) region, and the term TCR gamma constant domain refers to the extracellular TRGC region, or to a C-terminal truncated TRGC sequence. Likewise, the term “TCR delta variable domain” refers to the concatenation of the TCR delta V (TRDV) region without leader region (L) and the TCR delta D/J (TRDD/TRDJ) region, and the term “TCR delta constant domain” refers to the extracellular TRDC region, or to a C-terminal truncated TRDC sequence.

TCRs of the present disclosure preferably bind to a peptide-HLA molecule complex with a binding affinity (KD) of about 100 μM or less, about 50 μM or less, about 25 μM or less, or about 10 μM or less. More preferred are high affinity TCRs having binding affinities of about 1 μM or less, about 100 nM or less, about 50 nM or less, about 25 nM or less. Nonlimiting examples of preferred binding affinity ranges for TCRs of the present invention include about 1 nM to about 10 nM; about 10 nM to about 20 nM; about 20 nM to about 30 nM; about 30 nM to about 40 nM; about 40 nM to about 50 nM; about 50 nM to about 60 nM; about 60 nM to about 70 nM; about 70 nM to about 80 nM; about 80 nM to about 90 nM; and about 90 nM to about 100 nM.

As used herein in connect with TCRs of the present description, “specific binding” and grammatical variants thereof are used to mean a TCR having a binding affinity (KD) for a peptide-HLA molecule complex of 100 μM or less.

Alpha/beta heterodimeric TCRs of the present description may have an introduced disulfide bond between their constant domains. With or without the introduced inter-chain bond mentioned above, alpha/beta heterodimeric TCRs of the present description may have a TRAC constant domain sequence and a TRBC1 or TRBC2 constant domain sequence, and the TRAC constant domain sequence and the TRBC1 or TRBC2 constant domain sequence of the TCR may be linked by the native disulfide bond between Cys4 of exon 2 of TRAC and Cys2 of exon 2 of TRBC1 or TRBC2.

TCRs of the present disclosure may comprise a detectable label selected from the group consisting of a radionuclide, a fluorophore and biotin. TCRs of the present description may be conjugated to a therapeutically active agent, such as a radionuclide, a chemotherapeutic agent, or a toxin.

In an embodiment, a TCR of the present description having at least one mutation in the alpha chain and/or having at least one mutation in the beta chain has modified glycosylation compared to the unmutated TCR.

In an embodiment, a TCR comprising at least one mutation in the TCR alpha chain and/or TCR beta chain has a binding affinity for, and/or a binding half-life for, a peptide-HLA molecule complex, which is at least double that of a TCR comprising the unmutated TCR alpha chain and/or unmutated TCR beta chain. Affinity-enhancement of tumor-specific TCRs, and its exploitation, relies on the existence of a window for optimal TCR affinities. The existence of such a window is based on observations that TCRs specific for HLA-A2-restricted pathogens have KD values that are generally about 10-fold lower when compared to TCRs specific for HLA-A2-restricted tumor-associated self-antigens. It is now known, although tumor antigens have the potential to be immunogenic, because tumors arise from the individual's own cells only mutated proteins or proteins with altered translational processing will be seen as foreign by the immune system. Antigens that are upregulated or overexpressed (so called self-antigens) will not necessarily induce a functional immune response against the tumor: T-cells expressing TCRs that are highly reactive to these antigens will have been negatively selected within the thymus in a process known as central tolerance, meaning that only T-cells with low-affinity TCRs for self-antigens remain. Therefore, affinity of TCRs or variants of the present description to the peptides according to the present disclosure can be enhanced by methods well known in the art.

The present description further relates to a method of identifying and isolating a TCR according to the present description, said method comprising incubating PBMCs from HLA-A*02-negative healthy donors with A2/peptide monomers, incubating the PBMCs with tetramer-phycoerythrin (PE) and isolating the high avidity T-cells by fluorescence activated cell sorting (FACS)-Calibur analysis.

The present description further relates to a method of identifying and isolating a TCR according to the present description, said method comprising obtaining a transgenic mouse with the entire human TCRαβ gene loci (1.1 and 0.7 Mb), whose T-cells express a diverse human TCR repertoire that compensates for mouse TCR deficiency, immunizing the mouse with peptide of interest, incubating PBMCs obtained from the transgenic mice with tetramer-phycoerythrin (PE), and isolating the high avidity T-cells by fluorescence activated cell sorting (FACS)-Calibur analysis.

In one aspect, to obtain T-cells expressing TCRs of the present description, nucleic acids encoding TCR-alpha and/or TCR-beta chains of the present description are cloned into expression vectors, such as gamma retrovirus or lentivirus. The recombinant viruses are generated and then tested for functionality, such as antigen specificity and functional avidity. An aliquot of the final product is then used to transduce the target T-cell population (generally purified from patient PBMCs), which is expanded before infusion into the patient.

In another aspect, to obtain T-cells expressing TCRs of the present description, TCR RNAs are synthesized by techniques known in the art, e.g., in vitro transcription sys-tems. The in vitro-synthesized TCR RNAs are then introduced into primary CD8+ T-cells obtained from healthy donors by electroporation to re-express tumor specific TCR-alpha and/or TCR-beta chains.

To increase the expression, nucleic acids encoding TCRs of the present description may be operably linked to strong promoters, such as retroviral long terminal repeats (LTRs), cytomegalovirus (CMV), murine stem cell virus (MSCV) U3, phosphoglycerate kinase (PGK), 13-actin, ubiquitin, and a simian virus 40 (SV40)/CD43 composite promoter, elongation factor (EF)-1 a and the spleen focus-forming virus (SFFV) promoter. In a preferred embodiment, the promoter is heterologous to the nucleic acid being expressed.

In addition to strong promoters, TCR expression cassettes of the present description may contain additional elements that can enhance transgene expression, including a central polypurine tract (cPPT), which promotes the nuclear translocation of lentiviral constructs (Follenzi et al., 2000, which is incorporated by reference in its entirety), and the woodchuck hepatitis virus posttranscriptional regulatory element (wPRE), which increases the level of transgene expression by increasing RNA stability (Zufferey et al., 1999, which is incorporated by reference in its entirety).

The alpha and beta chains of a TCR of the present invention may be encoded by nucleic acids located in separate vectors, or may be encoded by polynucleotides located in the same vector.

Achieving high-level TCR surface expression requires that both the TCR-alpha and TCR-beta chains of the introduced TCR be transcribed at high levels. To do so, the TCR-alpha and TCR-beta chains of the present description may be cloned into bi-cistronic constructs in a single vector, which has been shown to be capable of overcoming this obstacle. The use of a viral intraribosomal entry site (IRES) between the TCR-alpha and TCR-beta chains results in the coordinated expression of both chains, because the TCR-alpha and TCR-beta chains are generated from a single transcript that is broken into two proteins during translation, ensuring that an equal molar ratio of TCR-alpha and TCR-beta chains are produced. (Schmitt et al. 2009, which is incorporated by reference in its entirety).

Nucleic acids encoding TCRs of the present description may be codon optimized to increase expression from a host cell. Redundancy in the genetic code allows some amino acids to be encoded by more than one codon, but certain codons are less “optimal” than others because of the relative availability of matching tRNAs as well as other factors (Gustafsson et al., 2004, which is incorporated by reference in its entirety). Modifying the TCR-alpha and TCR-beta gene sequences such that each amino acid is encoded by the optimal codon for mammalian gene expression, as well as eliminating mRNA instability motifs or cryptic splice sites, has been shown to significantly enhance TCR-alpha and TCR-beta gene expression (Scholten et al., 2006, which is incorporated by reference in its entirety).

Furthermore, mispairing between the introduced and endogenous TCR chains may result in the acquisition of specificities that pose a significant risk for autoimmunity. For example, the formation of mixed TCR dimers may reduce the number of CD3 molecules available to form properly paired TCR complexes, and therefore can significantly decrease the functional avidity of the cells expressing the introduced TCR (Kuball et al., 2007, which is incorporated by reference in its entirety).

To reduce mispairing, the C-terminus domain of the introduced TCR chains of the present description may be modified in order to promote interchain affinity, while de-creasing the ability of the introduced chains to pair with the endogenous TCR. These strategies may include replacing the human TCR-alpha and TCR-beta C-terminus domains with their murine counterparts (murinized C-terminus domain); generating a second interchain disulfide bond in the C-terminus domain by introducing a second cysteine residue into both the TCR-alpha and TCR-beta chains of the introduced TCR (cysteine modification); swapping interacting residues in the TCR-alpha and TCR-beta chain C-terminus domains (“knob-in-hole”); and fusing the variable domains of the TCR-alpha and TCR-beta chains directly to CD3s (CD3s fusion). (Schmitt et al. 2009, which is incorporated by reference in its entirety).

In an embodiment, a host cell is engineered to express a TCR of the present disclosure. In preferred embodiments, the host cell is a human T-cell or T-cell progenitor. In some embodiments the T-cell or T-cell progenitor is obtained from a cancer patient. In other embodiments the T-cell or T-cell progenitor is obtained from a healthy donor. Host cells of the present description can be allogeneic or autologous with respect to a patient to be treated. In one embodiment, the host is a gamma/delta T-cell transformed to express an alpha/beta TCR.

It is a further aspect of the invention to provide a method for producing a soluble T-cell receptor (sTCR) recognizing a specific peptide-MHC complex. Such soluble T-cell receptors can be generated from specific T-cell clones, and their affinity can be increased by mutagenesis targeting the complementarity-determining regions. For the purpose of T-cell receptor selection, phage display can be used (US 2010/0113300, Liddy et al. 2012, which is incorporated by reference in its entirety). For the purpose of stabilization of T-cell receptors during phage display and in case of practical use as drug, alpha and beta chain can be linked e.g. by non-native disulfide bonds, other covalent bonds (single-chain T-cell receptor), or by dimerization domains (Boulter et al., 2003; Card et al., 2004; Willcox et al., 1999, each of which are incorporated by reference in their entireties). The T-cell receptor can be linked to toxins, drugs, cytokines (see, for example, US 2013/0115191, which is incorporated by reference in its entirety), and domains recruiting effector cells such as an anti-CD3 domain, etc., in order to execute particular functions on target cells. Moreover, it could be expressed in T cells used for adoptive transfer. Further information can be found in WO 2004/033685A 1 and WO 2004/07 4322A1. A combination of sTCRs is described in WO 2012/056407A1, the contents of which are each incorporated by reference in their entireties). Further methods for the production are disclosed in WO 2013/057586A1, which is incorporated by reference in its entirety.

In addition, the peptides and/or the TCRs or antibodies or other binding molecules of the present invention can be used to verify a pathologist's diagnosis of a cancer based on a biopsied sample.

As used herein, the term “scaffold” refers to a molecule that specifically binds to an (e.g. antigenic) determinant. In one embodiment, a scaffold is able to direct the entity to which it is attached (e.g. a (second) antigen binding moiety) to a target site, for example to a specific type of tumor cell or tumor stroma bearing the antigenic determinant (e.g. the complex of an RNA editing-derived peptide, e.g., comprising, consisting of, or consisting essentially of one or more of the amino acid sequences selected from the group consisting of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9), with MHC, according to the application at hand). In another embodiment a scaffold is able to activate signaling through its target antigen, for example a T cell receptor complex antigen. Scaffolds include but are not limited to antibodies and fragments thereof, antigen binding domains of an antibody, comprising an antibody heavy chain variable region and an antibody light chain variable region, binding proteins comprising at least one ankyrin repeat motif and single domain antigen binding (SDAB) molecules, aptamers, (soluble) TCRs and (modified) cells such as allogenic or autologous T cells. To assess whether a molecule is a scaffold binding to a target, binding assays can be performed.

In an aspect, “specific” binding means that the scaffold binds the peptide-MHC-complex of interest better than other naturally occurring peptide-MHC-complexes, to an extent that a scaffold armed with an active molecule that is able to kill a cell bearing the specific target is not able to kill another cell without the specific target but presenting other peptide-MHC complex(es). Binding to other peptide-MHC complexes is irrelevant if the peptide of the cross-reactive peptide-MHC is not naturally occurring, i.e. not derived from the human HLA-peptidome. Tests to assess target cell killing are well known in the art. They should be performed using target cells (primary cells or cell lines) with unaltered peptide-MHC presentation, or cells loaded with peptides such that naturally occurring peptide-MHC levels are reached.

Each scaffold can comprise a labelling which provides that the bound scaffold can be detected by determining the presence or absence of a signal provided by the label. For example, the scaffold can be labelled with a fluorescent dye or any other applicable cellular marker molecule. Such marker molecules are well known in the art. For example, a fluorescence-labelling, for example provided by a fluorescence dye, can provide a visualization of the bound aptamer by fluorescence or laser scanning microscopy or flow cytometry.

Each scaffold can be conjugated with a second active molecule such as for example IL-21, anti-CD3, and anti-CD28.

For further information on polypeptide scaffolds see for example the background section of WO 20141071978A1, which is incorporated by reference in its entirety.

The present disclosure further relates to aptamers that bind to an RNA editing-derived peptide, e.g., comprising, consisting of, or consisting essentially of one or more of the amino acid sequences selected from the group consisting of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9) or bind to an MHC molecule/RNA editing-derived peptide complex. Aptamers (see for example WO 2014/191359 and the literature as cited therein, which is incorporated by reference in its entirety) are short single-stranded nucleic acid molecules, which can fold into defined three-dimensional structures and recognize specific target structures. They have appeared to be suitable alternatives for developing targeted therapies. Aptamers have been shown to selectively bind to a variety of complex targets with high affinity and specificity.

Aptamers recognizing cell surface located molecules have been identified within the past decade and provide means for developing diagnostic and therapeutic approaches. Since aptamers have been shown to possess almost no toxicity and immunogenicity they are promising candidates for biomedical applications. Indeed aptamers, for example prostate-specific membrane-antigen recognizing aptamers, have been successfully employed for targeted therapies and shown to be functional in xenograft in vivo models. Furthermore, aptamers recognizing specific tumor cell lines have been identified.

DNA aptamers can be selected to reveal broad-spectrum recognition properties for various cancer cells, and particularly those derived from solid tumors, while nontumorigenic and primary healthy cells are not recognized. If the identified aptamers recognize not only a specific tumor sub-type but rather interact with a series of tumors, this renders the aptamers applicable as so-called broad-spectrum diagnostics and therapeutics.

Further, investigation of cell-binding behavior with flow cytometry showed that the aptamers revealed very good apparent affinities that are within the nanomolar range.

Aptamers are useful for diagnostic and therapeutic purposes. Further, it could be shown that some of the aptamers are taken up by tumor cells and thus can function as molecular vehicles for the targeted delivery of anti-cancer agents such as siRNA into tumor cells.

Aptamers can be selected against complex targets, such as cells and tissues and complexes of the peptides comprising, preferably consisting of, a sequence according to any of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), and SPRQPPLLL (SEQ ID NO: 9), according to the invention at hand with the MHC molecule, using the cell-SELEX (Systematic Evolution of Ligands by Exponential enrichment) technique.

The antibodies or TCRs may also be used for in vivo diagnostic assays.

Generally, the antibody is labeled with a radionucleotide (such as ¹¹¹In, ⁹⁹Tc, ¹⁴C, ¹³¹I, ³H, ³²P or ³⁵S) so that the tumor can be localized using immunoscintiography. In one embodiment, antibodies or fragments thereof bind to the extracellular domains of two or more targets of a protein selected from the group consisting of the above-mentioned proteins, and the affinity value (Kd) is less than 1×10 μM.

Antibodies for diagnostic use may be labeled with probes suitable for detection by various imaging methods. Methods for detection of probes include, but are not limited to, fluorescence, light, confocal and electron microscopy; magnetic resonance imaging and spectrometry; fluoroscopy, computed tomography and positron emission tomography. Suitable probes include, but are not limited to, fluorescein, rhodamine, eosin and other fluorophores, radioisotopes, gold, gadolinium and other lanthanides, paramagnetic iron, fluorine-18 and other positron-emitting radionuclides. Additionally, probes may be bi- or multi-functional and be detectable by more than one of the methods listed. These antibodies may be directly or indirectly labeled with said probes. Attachment of probes to the antibodies includes covalent attachment of the probe, incorporation of the probe into the antibody, and the covalent attachment of a chelating compound for binding of probe, amongst others well recognized in the art. For immunohistochemistry, the disease tissue sample may be fresh or frozen or may be embedded in paraffin and fixed with a preservative such as formalin. The fixed or embedded section contains the sample are contacted with a labeled primary antibody and secondary antibody, wherein the antibody is used to detect the expression of the proteins in situ.

The present disclosure further relates to methods wherein an antigen is loaded onto class I or II MHC molecules expressed on the surface of a suitable antigen-presenting cell or artificial antigen-presenting cell. In another aspect, an antigen is loaded onto class I or II MHC molecules expressed on the surface of a suitable antigen-presenting cell or artificial antigen-presenting cell by contacting a sufficient amount of the antigen with an antigen-presenting cell.

The present disclosure further relates to activated T cells, produced by the method according to the present disclosure, wherein the T cell selectively recognizes a cell which expresses a peptide comprising an amino acid sequence according to the present disclosure.

In an aspect, for an MHC class I peptide including RNA editing-derived epitopes to trigger (elicit) a cellular immune response, it also must bind to an MHC-molecule. This process is dependent on the allele of the MHC-molecule and specific polymorphisms of the amino acid sequence of the peptide. MHC-class-I-binding peptides are usually 8-12 amino acid residues in length and usually contain two conserved residues (“anchors”) in their sequence that interact with the corresponding binding groove of the MHC-molecule. In this way each MHC allele has a “binding motif” determining which peptides can bind specifically to the binding groove.

In another aspect, in an MHC class I dependent immune reaction, peptides, such as RNA editing-derived epitopes, bind to certain MHC class I molecules expressed by tumor cells and are subsequently recognized by T cells bearing specific T cell receptors (TCR).

For proteins to be recognized by T-lymphocytes as tumor-specific or -associated antigens, and to be used in a therapy, certain prerequisites are generally fulfilled. The antigen, such as RNA editing-derived epitopes, should be expressed mainly by tumor cells and not, or in comparably small amounts, by normal healthy tissues. In a preferred embodiment, the peptide, such as RNA editing-derived epitopes, should be over-presented by tumor cells as compared to normal healthy tissues. It is furthermore desirable that the respective antigen is not only present in a type of tumor, but also in high concentrations (i.e. copy numbers of the respective peptide per cell). Tumor-specific and tumor-associated antigens are often derived from proteins directly involved in transformation of a normal cell to a tumor cell due to their function, e.g. in cell cycle control or suppression of apoptosis. Additionally, downstream targets of the proteins directly causative for a transformation may be up-regulated and thus may be indirectly tumor-associated. Such indirect tumor-associated antigens may also be targets of a vaccination approach (Singh-Jasuja et al., 2004). It is essential that epitopes including RNA editing-derived epitopes are present in the amino acid sequence of the antigen, in order to ensure that such a peptide (“immunogenic peptide”), being derived from a tumor associated antigen, leads to an in vitro or in vivo T cell-response.

In an aspect, any peptide including RNA editing-derived epitopes able to bind an MHC molecule may function as a T-cell epitope. A prerequisite for the induction of an in vitro or in vivo T-cell-response is the presence of a T cell having a corresponding TCR and the absence of immunological tolerance for a particular epitope.

Therefore, tumor associated antigens, such as RNA editing-derived epitopes, may be a starting point for the development of a T cell based therapy including but not limited to tumor vaccines. The methods for identifying and characterizing the tumor associated antigens are usually based on the use of T-cells that can be isolated from patients or healthy subjects or they are based on the generation of differential transcription profiles or differential peptide expression patterns between tumors and normal tissues. However, the identification of RNA editing-derived epitopes may be based on the use of tumor tissues or human tumor cell lines that have ADAR activity, such that these ADAR-positive tumor tissues or tumor cell lines may express RNA edited proteins that, in turn, present RNA edited peptide in a complex with MHC molecules on the cell surface. In a preferred embodiment of the present disclosure, it is therefore important to select only those RNA editing-derived epitopes against which a functional and/or a proliferating T cell can be found. Such a functional T cell is defined as a T cell, which upon stimulation with a specific RNA editing-derived epitope can be clonally expanded and is able to execute effector functions (“effector T cell”).

In case of targeting RNA editing-derived epitope-MHC by specific TCRs (e.g. soluble TCRs) according to the present disclosure, the immunogenicity of the underlying peptides is secondary. In these cases, the presentation is the determining factor.

The term “T-cell response” means the specific proliferation and activation of effector functions induced by a peptide, e.g., RNA editing-derived epitope, in vitro or in vivo. For MHC class I restricted cytotoxic T cells, effector functions may be lysis of peptide-pulsed, peptide-precursor pulsed or naturally peptide-presenting target cells, secretion of cytokines, preferably Interferon-gamma, TNF-alpha, or IL-2 induced by peptide, secretion of effector molecules, preferably granzymes or perforins induced by peptide, or degranulation.

The term “peptide” is used herein to designate a series of amino acid residues, connected one to the other typically by peptide bonds between the alpha-amino and carbonyl groups of the adjacent amino acids. The peptides are preferably 9 amino acids in length, but can be as short as 8 amino acids in length, and as long as 10, 11, or 12 or longer, and in case of MHC class II peptides (elongated variants of the peptides of the disclosure) they can be as long as 13, 14, 15, 16, 17, 18, 19 or 20 or more amino acids in length. In an aspect, peptides described herein are from 8 to 100 amino acids, from 8 to 30 amino acids, from 8 to 16 amino acids, from 8 and 14 amino acids, from 8 to 12 amino acids, from 8 to 10 amino acids, from 9 to 15 amino acids, from 9 to 14 amino acids, from 9 to 13 amino acids, from 9 to 12 amino acids, from 9 to 11 amino acids; from 10 to 15 amino acids, from 10 to 14 amino acids, from 10 to 13 amino acids, or from 10 to 12 amino acids.

Furthermore, the term “peptide” shall include salts of a series of amino acid residues, connected one to the other typically by peptide bonds between the alpha-amino and carbonyl groups of the adjacent amino acids. Preferably, the salts are pharmaceutical acceptable salts of the peptides, such as, for example, the chloride or acetate (trifluoro-acetate) salts. It may be noted that the salts of the peptides according to the present disclosure differ substantially from the peptides in their state(s) in vivo, as the peptides are not salts in vivo.

The term “peptide” shall also include “oligopeptide”. The term “oligopeptide” is used herein to designate a series of amino acid residues, connected one to the other typically by peptide bonds between the alpha-amino and carbonyl groups of the adjacent amino acids. The length of the oligopeptide is not critical to the disclosure, as long as the correct epitope or epitopes are maintained therein. The oligopeptides are typically less than about 30 amino acid residues in length, and greater than about 15 amino acids in length.

The term “polypeptide” designates a series of amino acid residues, connected one to the other typically by peptide bonds between the alpha-amino and carbonyl groups of the adjacent amino acids. The length of the polypeptide is not critical to the disclosure as long as the correct epitopes are maintained. In contrast to the terms peptide or oligopeptide, the term polypeptide is meant to refer to molecules containing more than about 30 amino acid residues.

A peptide, oligopeptide, protein or polynucleotide coding for such a molecule is “immunogenic” (and thus is an “immunogen” within the present disclosure), if it is capable of inducing an immune response. In the case of the present disclosure, immunogenicity is more specifically defined as the ability to induce a T-cell response. Thus, an “immunogen” would be a molecule that is capable of inducing an immune response, and in the case of the present disclosure, a molecule capable of inducing a T-cell response. In an-other aspect, the immunogen can be the peptide, the complex of the peptide with MHC, oligopeptide, and/or protein that is used to raise specific antibodies or TCRs against it.

A class I T cell “epitope” requires a short peptide, such as RNA editing-derived peptide, that is bound to a class I MHC receptor, forming a ternary complex (MHC class I alpha chain, beta-2-microglobulin, and peptide) that can be recognized by a T cell bearing a matching T-cell receptor binding to the MHC/peptide complex with appropriate affinity. Peptides binding to MHC class I molecules are typically 8-14 amino acids in length, and most typically 9 amino acids in length.

Generally, peptides and variants (at least those containing peptide linkages between amino acid residues) may be synthesized by the Fmoc-polyamide mode of solid-phase peptide synthesis as disclosed by Lukas et al. (Lukas et al., 1981, which is incorporated by reference in its entirety) and by references as cited therein. Temporary N-amino group protection is afforded by the 9-fluorenylmethyloxycarbonyl (Fmoc) group. Repetitive cleavage of this highly base-labile protecting group is done using 20% piperidine in N, N-dimethylformamide. Side-chain functionalities may be protected as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine). Where glutamine or asparagine are C-terminal residues, use is made of the 4,4′-dimethoxybenzhydryl group for protection of the side chain amido functionalities. The solid-phase support is based on a polydimethyl-acrylamide polymer constituted from the three monomers dimethylacrylamide (backbone-monomer), bisacryloylethylene diamine (cross linker) and acryloylsarcosine methyl ester (functionalizing agent). The peptide-to-resin cleavable linked agent used is the acid-labile 4-hydroxymethyl-phenoxyacetic acid derivative. All amino acid derivatives are added as their preformed symmetrical anhydride derivatives with the exception of asparagine and glutamine, which are added using a reversed N, N-dicyclohexyl-carbodiimide/1hydroxybenzotriazole mediated coupling procedure. All coupling and deprotection reactions are monitored using ninhydrin, trinitrobenzene sulphonic acid or isotin test procedures. Upon completion of synthesis, peptides are cleaved from the resin support with concomitant removal of side-chain protecting groups by treatment with 95% trifluoroacetic acid containing a 50% scavenger mix. Scavengers commonly used include ethanedithiol, phenol, anisole and water, the exact choice depending on the constituent amino acids of the peptide being synthesized. Also, a combination of solid phase and solution phase methodologies for the synthesis of peptides is possible (see, for example, Bruckdorfer et al., 2004, which is incorporated by reference in its entirety).

Trifluoroacetic acid is removed by evaporation in vacuo, with subsequent trituration with diethyl ether affording the crude peptide. Any scavengers present are removed by a simple extraction procedure which on lyophilization of the aqueous phase affords the crude peptide free of scavengers. Reagents for peptide synthesis are generally available from e.g. Calbiochem-Novabiochem (Nottingham, UK).

Purification may be performed by any one, or a combination of, techniques such as re-crystallization, size exclusion chromatography, ion-exchange chromatography, hydrophobic interaction chromatography and (usually) reverse-phase high performance liquid chromatography using e.g. acetonitrile/water gradient separation.

Analysis of peptides may be carried out using thin layer chromatography, electrophoresis, in particular capillary electrophoresis, solid phase extraction (CSPE), reverse-phase high performance liquid chromatography, amino-acid analysis after acid hydrolysis and by fast atom bombardment (FAB) mass spectrometric analysis, as well as MALDI and ESI-Q-TOF mass spectrometric analysis.

For the identification of peptides of the present disclosure, two databases of RNA expression data were compared together: RNASeq tumor data generated by the TCGA Research Network (cancergenome.nih.gov/) and RNASeq data (GTEx) covering around 3000 normal (healthy) tissue samples (Lonsdale, 2013, which is incorporated by reference in its entirety). Genes were screened, with were over-expressed in tumor tissues samples compared with the normal (healthy) tissue samples. Then, cancer-associated peptides derived from the protein products of these genes were identified by mass spectrometry using the XPRESIDENT™ platform as described herein.

In order to select over-presented peptides, a presentation profile is calculated showing the median sample presentation as well as replicate variation. The profile juxtaposes samples of the tumor entity of interest to a baseline of normal tissue samples. Each of these profiles can then be consolidated into an over-presentation score by calculating the p-value of a Linear Mixed-Effects Model (Pinheiro et al., 2015, which is incorporated by reference in its entirety) adjusting for multiple testing by False Discovery Rate (Benjamini and Hochberg, 1995, which is incorporated by reference in its entirety).

For the identification and relative quantitation of HLA ligands by mass spectrometry, HLA molecules from shock-frozen tissue samples were purified and HLA-associated peptides were isolated. The isolated peptides were separated and sequences were identified by online nano-electrospray-ionization (nanoESI) liquid chromatography-mass spectrometry (LC-MS) experiments. The resulting peptide sequences were verified by comparison of the fragmentation pattern of natural tumor-associated peptides (TUMAPs) recorded from cancer samples (N=450 A*02-positive samples, N=211 A*24-positive samples) with the fragmentation patterns of corresponding synthetic reference peptides of identical sequences. Since the peptides were directly identified as ligands of HLA molecules of primary tumors, these results provide direct evidence for the natural processing and presentation of the identified peptides on primary cancer tissue obtained from A*02 and/or A*24-positive cancer patients.

The discovery pipeline XPRESIDENT® v2.1 (see, for example, US 2013-0096016, which is hereby incorporated by reference in its entirety) allows the identification and selection of relevant over-presented peptide vaccine candidates based on direct relative quantitation of HLA-restricted peptide levels on cancer tissues in comparison to several different non-cancerous tissues and organs. This was achieved by the development of label-free differential quantitation using the acquired LC-MS data processed by a proprietary data analysis pipeline, combining algorithms for sequence identification, spectral clustering, ion counting, retention time alignment, charge state deconvolution and normalization.

Presentation levels including error estimates for each peptide and sample were established. Peptides exclusively presented on tumor tissue and peptides over-presented in tumor versus non-cancerous tissues and organs have been identified.

HLA-peptide complexes from tissue samples were purified and HLA-associated peptides were isolated and analyzed by MS/MS and/or LC-MS (see examples). All RNA editing-derived epitopes contained in the present application were identified with this approach on primary cancer samples confirming their presentation on primary glioblastoma, breast cancer, colorectal cancer, renal cell carcinoma, chronic lymphocytic leukemia, hepatocellular carcinoma, non-small cell and small cell lung cancer, Non-Hodgkin lymphoma, acute myeloid leukemia, ovarian cancer, pancreatic cancer, prostate cancer, esophageal cancer including cancer of the gastric-esophageal junction, gallbladder cancer and cholangiocarcinoma, melanoma, gastric cancer, urinary bladder cancer, or uterine cancer.

RNA editing-derived epitopes identified on multiple cancer and normal tissues were quantified using ion-counting of label-free MS/MS and/or LC-MS data. The method assumes that MS/MS and/or LC-MS signal areas of a peptide correlate with its abundance in the sample. All quantitative signals of a peptide in various MS/MS and/or LC-MS experiments were normalized based on central tendency, averaged per sample and merged into a bar plot, called presentation profile. The presentation profile consolidates different analysis methods like protein database search, spectral clustering, charge state deconvolution (decharging) and retention time alignment and normalization.

Furthermore, the discovery pipeline XPRESIDENT® v2.1 allows the direct absolute quantitation of MHC-, preferably HLA-restricted, peptide levels on cancer or other infected tissues. Briefly, the total cell count was calculated from the total DNA content of the analyzed tissue sample. The total peptide amount for an RNA editing-derived epitope in a tissue sample was measured by nanoLC-MS/MS as the ratio of the natural TUMAP and a known amount of an isotope-labelled version of the TUMAP, the so-called internal standard. The efficiency of RNA editing-derived epitope isolation was determined by spiking peptide:MHC complexes of all selected TUMAPs into the tissue lysate at the earliest possible point of the RNA editing-derived epitope isolation procedure and their detection by nanoLC-MS/MS following completion of the peptide isolation procedure. The total cell count and the amount of total peptide were calculated from triplicate measurements per tissue sample. The peptide-specific isolation efficiencies were calculated as an average from 10 spike experiments each measured as a triplicate.

Besides over-presentation of the peptide, mRNA expression of the underlying gene was tested. mRNA data were obtained via RNASeq analyses of normal tissues and cancer tissues. An additional source of normal tissue data was a database of publicly available RNA expression data from around 3000 normal tissue samples (Lonsdale, 2013, which is incorporated by reference in its entirety). Peptides which are derived from proteins whose coding mRNA is highly expressed in cancer tissue, but very low or absent in vital normal tissues, were preferably included in the present disclosure.

The present disclosure provides peptides that are useful in treating cancers/tumors, preferably glioblastoma, breast cancer, colorectal cancer, renal cell carcinoma, chronic lymphocytic leukemia, hepatocellular carcinoma, non-small cell and small cell lung cancer, Non-Hodgkin lymphoma, acute myeloid leukemia, ovarian cancer, pancreatic cancer, prostate cancer, esophageal cancer including cancer of the gastric-esophageal junction, gallbladder cancer and cholangiocarcinoma, melanoma, gastric cancer, urinary bladder cancer, head and neck squamous cell carcinoma, and uterine cancer that over- or exclusively present the peptides of the disclosure. These peptides were shown by mass spectrometry to be naturally presented by HLA molecules on primary human cancer samples.

Many of the source gene/proteins (also designated “full-length proteins” or “underlying proteins”) from which the peptides are derived were shown to be highly over-expressed in cancer compared with normal tissues—“normal tissues” in relation to this disclosure shall mean either healthy cells or tissue derived from the same organ as the tumor, or other normal tissue cells, demonstrating a high degree of tumor association of the source genes. Moreover, the peptides themselves are strongly over-presented on tumor tissue—“tumor tissue” in relation to this disclosure shall mean a sample from a patient suffering from cancer, but not on normal tissues (see Example 1).

HLA-bound peptides can be recognized by the immune system, specifically T lymphocytes. T cells can destroy the cells presenting the recognized HLA/peptide complex, e.g. glioblastoma, breast cancer, colorectal cancer, renal cell carcinoma, chronic lymphocytic leukemia, hepatocellular carcinoma, non-small cell and small cell lung cancer, Non-Hodgkin lymphoma, acute myeloid leukemia, ovarian cancer, pancreatic cancer, prostate cancer, esophageal cancer including cancer of the gastric-esophageal junction, gallbladder cancer and cholangiocarcinoma, melanoma, gastric cancer, urinary bladder cancer, or uterine cancer cells presenting the derived peptides.

A “pharmaceutical composition” is a composition suitable for administration to a human being in a medical setting. Preferably, a pharmaceutical composition is sterile and produced according to GMP guidelines.

An embodiment of the present invention thus relates to a non-naturally occurring molecule according to the invention that has been synthetically produced (e.g. synthesized) as a pharmaceutically acceptable salt. Methods to synthetically produce peptides and/or polypeptides are well known in the art. The salts of the molecules according to the present invention differ substantially from the molecules in their state(s) in vivo, as the molecules as generated in vivo are no salts. Preferably, the salts are pharmaceutically acceptable salts of the molecules. These salts according to the invention include alkaline and earth alkaline salts such as salts of the Hofmeister series comprising as anions PO₄³⁻, SO₄²⁻, CH₃COO⁻, Cl⁻, Br, NO₃⁻, ClO₄⁻, SCN⁻ and as cations NH₄⁺, Rb⁺, K⁺, Na⁺, Cs⁺, Li⁺, Zn²⁺, Mg²⁺, Ca²⁺, Mn²⁺, Cu²⁺ and Ba²⁺. Particularly salts are selected from (NH₄)₃PO₄, (NH₄)₂HPO₄, (NH₄)H₂PO₄, (NH₄)₂SO₄, NH₄CH₃COO, NH₄Cl, NH₄Br, NH₄NO₃, NH₄ClO₄, NH₄I, NH₄SCN, Rb₃PO₄, Rb₂HPO₄, RbH₂PO₄, Rb₂SO₄, Rb₄CH₃COO, Rb₄Cl, Rb₄Br, Rb₄NO₃, Rb₄ClO₄, Rb₄I, Rb₄SCN, K₃PO₄, K₂HPO₄, KH₂PO₄, K₂SO₄, KCH₃COO, KCl, KBr, KNO₃, KClO₄, KI, KSCN, Na₃PO₄, Na₂HPO₄, NaH₂PO₄, Na₂SO₄, NaCH₃COO, NaCl, NaBr, NaNO₃, NaClO₄, NaI, NaSCN, ZnCl₂ Cs₃PO₄, Cs₂HPO₄, CsH₂PO₄, Cs₂SO₄, CsCH₃COO, CsCl, CsBr, CsNO₃, CsClO₄, CsI, CsSCN, Li₃PO₄, Li₂HPO₄, LiH₂PO₄, Li₂SO₄, LiCH₃COO, LiCl, LiBr, LiNO₃, LiClO₄, LiI, LiSCN, Cu₂SO₄, Mg₃(PO₄)₂, Mg₂HPO₄, Mg(H₂PO₄)₂, Mg₂SO₄, Mg(CH₃COO)₂, MgCl₂, MgBr₂, Mg(NO₃)₂, Mg(ClO₄)₂, MgI₂, Mg(SCN)₂, MnCl₂, Ca₃(PO₄) Ca₂HPO₄, Ca(H₂PO₄)₂, CaSO₄, Ca(CH₃COO)₂, CaCl₂, CaBr₂, Ca(NO₃)₂, Ca(ClO₄)₂, CaI₂, Ca(SCN)₂, Ba₃(PO₄)₂, Ba₂HPO₄, Ba(H₂PO₄)₂, BaSO₄, Ba(CH₃COO)₂, BaCl₂, BaBr₂, Ba(NO₃)₂, Ba(ClO₄)₂, BaI₂, and Ba(SCN)₂. Particularly preferred are NH acetate, MgCl₂, KH₂PO₄, Na₂SO₄, KCl, NaCl, and CaCl₂, such as, for example, the chloride or acetate (trifluoroacetate) salts.

In a preferred embodiment, the pharmaceutical compositions comprise the peptides as salts of acetic acid (acetates), trifluoro acetates or hydrochloric acid (chlorides).

In an aspect, a polypeptide described herein is in the form of a pharmaceutically acceptable salt. In another aspect, a polypeptide in the form of a pharmaceutical salt is in crystalline form.

In an aspect, a pharmaceutically acceptable salt described herein refers to salts which possess toxicity profiles within a range that is acceptable for pharmaceutical applications.

As used herein, “a pharmaceutically acceptable salt” refers to a derivative of the disclosed peptides wherein the peptide is modified by making acid or base salts of the agent. For example, acid salts are prepared from the free base (typically wherein the neutral form of the drug has a neutral —NH2 group) involving reaction with a suitable acid.

Suitable acids for preparing acid salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluenesulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a peptide are prepared using a pharmaceutically acceptable base such as sodium hydroxide, potassium hydroxide, ammonium hydroxide, calcium hydroxide, trimethylamine or the like.

In an aspect, pharmaceutically acceptable salts may increase the solubility and/or stability of peptides of described herein. In another aspect, pharmaceutical salts described herein may be prepared by conventional means from the corresponding carrier peptide or complex by reacting, for example, the appropriate acid or base with peptides or complexes as described herein. In another aspect, the pharmaceutically acceptable salts are in crystalline form or semi-crystalline form. In yet another aspect, pharmaceutically acceptable salts may include, for example, those described in Handbook of Pharmaceutical Salts: Properties, Selection, and Use By P. H. Stahl and C. G. Wermuth (Wiley-VCH 2002) and L. D. Bighley, S. M. Berge, D. C. Monkhouse, in “Encyclopedia of Pharmaceutical Technology”. Eds. J. Swarbrick and J. C. Boylan, Vol. 13, Marcel Dekker, Inc., New York, Basel, Hong Kong 1995, pp. 453-499, each of these references is herein incorporated by reference in their entirety.

Preferably, the medicament of the present disclosure is an immunotherapeutic such as a vaccine. It may be administered directly into the patient, into the affected organ or systemically i.d., i.m., s.c., i.p. and i.v., or applied ex vivo to cells derived from the patient or a human cell line which are subsequently administered to the patient, or used in vitro to select a subpopulation of immune cells derived from the patient, which are then re-administered to the patient. If the nucleic acid is administered to cells in vitro, it may be useful for the cells to be transfected so as to co-express immune-stimulating cytokines, such as interleukin-2. The peptide may be substantially pure, or combined with an immune-stimulating adjuvant (see below) or used in combination with immune-stimulatory cytokines, or be administered with a suitable delivery system, for example liposomes. The peptide may also be conjugated to a suitable carrier such as keyhole limpet haemocyanin (KLH) or mannan (see WO 95/18145 and (Longenecker et al., 1993), which is incorporated by reference in its entirety). The peptide may also be tagged, may be a fusion protein, or may be a hybrid molecule. The peptides whose sequence is given in the present disclosure are expected to stimulate CD4 or CD8 T cells. However, stimulation of CD8 T cells is more efficient in the presence of help provided by CD4 T-helper cells. Thus, for MHC Class I epitopes that stimulate CD8 T cells the fusion partner or sections of a hybrid molecule suitably provide epitopes which stimulate CD4-positive T cells. CD4- and CD8-stimulating epitopes are well known in the art and include those identified in the pre-sent disclosure.

The medicament of the disclosure may also include one or more adjuvants. Adjuvants are substances that non-specifically enhance or potentiate the immune response (e.g., immune responses mediated by CD8-positive T cells and helper-T (TH) cells to an anti-gen, and would thus be considered useful in the medicament of the present disclosure. Suitable adjuvants include, but are not limited to, 1018 ISS, aluminum salts, AMPLIVAX®, AS15, BCG, CP-870,893, CpG7909, CyaA, dSLIM, flagellin or TLR5 ligands derived from flagellin, FLT3 ligand, GM-CSF, IC30, IC31, Imiquimod (ALDARA®), resiquimod, ImuFact IMP321, Interleukins as IL-2, IL-13, IL-21, Interferon-alpha or -beta, or pegylated derivatives thereof, IS Patch, ISS, ISCOMATRIX, ISCOMs, JuvImmune®, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, OspA, PepTel® vector system, poly(lactid co-glycolid) [PLG]-based and dextran microparticles, talactoferrin SRL172, Virosomes and other Virus-like particles, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Adjuvants such as Freund's or GM-CSF are preferred. Several immuno-logical adjuvants (e.g., MF59) specific for dendritic cells and their preparation have been described previously (Allison and Krummel, 1995, which is incorporated by reference in its entirety). Also, cytokines may be used. Several cytokines have been directly linked to influencing dendritic cell migration to lymphoid tissues (e.g., TNF-), accelerating the maturation of dendritic cells into efficient antigen-presenting cells for T-lymphocytes (e.g., GM-CSF, IL-1 and IL-4) (U.S. Pat. No. 5,849,589, incorporated herein by reference in its entirety) and acting as immunoadjuvants (e.g., IL-12, IL-15, IL-23, IL-7, IFN-alpha. IFN-beta) (Gabrilovich et al., 1996, which is incorporated by reference in its entirety).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly it enhances dendritic cell maturation and differentiation, resulting in enhanced activation of TH1 cells and strong cytotoxic T-lymphocyte (CTL) generation, even in the absence of CD4 T cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG in some experiments (Krieg, 2006, which is incorporated by reference in its entirety). U.S. Pat. No. 6,406,705 B1, which is incorporated by reference in its entirety, describes the combined use of CpG oligonucleotides, non-nucleic acid adjuvants and an antigen to induce an antigen-specific immune response. A CpG TLR9 antagonist is dSLIM (double Stem Loop Immunomodulator) by Mologen (Berlin, Germany) which is a preferred component of the pharmaceutical composition of the present disclosure. Other TLR binding molecules such as RNA binding TLR 7, TLR 8 and/or TLR 9 may also be used.

Other examples for useful adjuvants include, but are not limited to chemically modified CpGs (e.g. CpR, Idera), dsRNA analogues such as Poly(I:C) and derivates thereof (e.g. AmpliGen®, Hiltonol®, poly-(ICLC), poly(IC-R), poly(I:C12U), non-CpG bacterial DNA or RNA as well as immunoactive small molecules and antibodies such as cyclophosphamide, sunitinib, immune checkpoint inhibitors including ipilimumab, nivolumab, pembrolizumab, atezolizumab, avelumab, durvalumab, and cemiplimab, Bevacizumab®, celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system (e.g. anti-CD40, anti-TGFbeta, anti-TNFalpha receptor) and SC58175, which may act therapeutically and/or as an adjuvant. The amounts and concentrations of adjuvants and additives useful in the context of the present disclosure can readily be determined by the skilled artisan without undue experimentation.

Preferred adjuvants are anti-CD40, imiquimod, resiquimod, GM-CSF, cyclophosphamide, sunitinib, atezolizumab, bevacizumab, interferon-alpha, interferon-beta, CpG oligonucleotides and derivatives, poly-(I:C) and derivatives, RNA, sildenafil, particulate formulations with poly(lactide co-glycolide) (PLG), virosomes, interleukin (IL)-1, IL-2, IL-4, IL-7, IL-12, IL-13, IL-15, IL-21, and/or IL-23.

In a preferred embodiment, the pharmaceutical composition according to the disclosure the adjuvant is selected from the group consisting of colony-stimulating factors, such as Granulocyte Macrophage Colony Stimulating Factor (GM-CSF, sargramostim), cyclophosphamide, imiquimod, resiquimod, and interferon-alpha.

In a preferred embodiment, the pharmaceutical composition according to the disclosure the adjuvant is selected from the group consisting of colony-stimulating factors, such as Granulocyte Macrophage Colony Stimulating Factor (GM-CSF, sargramostim), cyclophosphamide, imiquimod and resiquimod. In a preferred embodiment of the pharmaceutical composition according to the disclosure, the adjuvant is cyclophosphamide, imiquimod or resiquimod. Even more preferred adjuvants are Montanide IMS 1312, Montanide ISA 206, Montanide ISA 50V, Montanide ISA-51, poly-ICLC (Hiltonol®) and anti-CD40 mAB, or combinations thereof.

This composition is used for parenteral administration, such as subcutaneous, intra-dermal, intramuscular or oral administration. For this, the peptides and optionally other molecules are dissolved or suspended in a pharmaceutically acceptable, preferably aqueous carrier. In addition, the composition can contain excipients, such as buffers, binding agents, blasting agents, diluents, flavors, lubricants, etc. The peptides can also be administered together with immune stimulating substances, such as cytokines. An extensive listing of excipients that can be used in such a composition, can be, for ex-ample, taken from A. Kibbe, Handbook of Pharmaceutical Excipients (Kibbe, 2000, which is incorporated by reference in its entirety). The composition can be used for a prevention, prophylaxis and/or therapy of adenomatous or cancerous diseases. Exemplary formulations can be found in, for example, EP2112253, which is incorporated by reference in its entirety.

The immune response triggered by the vaccine according to the disclosure attacks the cancer in different cell-stages and different stages of development. Furthermore, different cancer associated signaling pathways are attacked. This is an advantage over vaccines that address only one or few targets, which may cause the tumor to easily adapt to the attack (tumor escape). Furthermore, not all individual tumors express the same pattern of antigens. Therefore, a combination of several tumor-associated peptides ensures that every single tumor bears at least some of the tar-gets. The composition is designed in such a way that each tumor is expected to ex-press several of the antigens and cover several independent pathways necessary for tumor growth and maintenance. Thus, the vaccine can easily be used “off-the-shelf” for a larger patient population. This means that a pre-selection of patients to be treated with the vaccine can be restricted to HLA typing, does not require any additional biomarker assessments for antigen expression, but it is still ensured that several targets are simultaneously attacked by the induced immune response, which is important for efficacy (Banchereau et al., 2001; Walter et al., 2012, which is incorporated by reference in its entirety).

The present disclosure further relates to the peptides according to the disclosure, where-in the peptide is part of a fusion protein, in particular comprising N-terminal amino acids of the HLA-DR antigen-associated invariant chain (Ii), or wherein the peptide is fused to (or into) an antibody, such as, for example, an antibody that is specific for dendritic cells.

Another aspect of the present disclosure includes an in vitro method for producing activated T cells, the method comprising contacting in vitro T cells with antigen loaded human MHC molecules expressed on the surface of a suitable antigen-presenting cell for a period of time sufficient to activate the T cell in an antigen specific manner, wherein the antigen is a peptide according to the disclosure. Preferably a sufficient amount of the antigen is used with an antigen-presenting cell.

Preferably the mammalian cell lacks or has a reduced level or function of the TAP pep-tide transporter. Suitable cells that lack the TAP peptide transporter include T2, RMA-S and Drosophila cells. TAP is the transporter associated with antigen processing.

The human peptide loading deficient cell line T2 is available from the American Type Culture Collection, 12301 Parklawn Drive, Rockville, Md. 20852, USA under Catalogue No CRL 1992; the Drosophila cell line Schneider line 2 is available from the ATCC under Catalogue No CRL 19863; the mouse RMA-S cell line is described in Ljunggren et al. (Ljunggren and Karre, 1985, which is incorporated by reference in its entirety).

Preferably, before transfection the host cell expresses substantially no MHC class I molecules. It is also preferred that the stimulator cell expresses a molecule important for providing a co-stimulatory signal for T-cells such as any of B7.1, B7.2, ICAM-1 and LFA 3. The nucleic acid sequences of numerous MHC class I molecules and of the co-stimulator molecules are publicly available from the GenBank and EMBL databases.

In case of an MHC class I RNA editing-derived epitope being used as an antigen, the T cells are CD8-positive T cells.

If an antigen-presenting cell is transfected to express such an RNA editing-derived epitope, preferably the cell comprises an expression vector capable of expressing a peptide containing RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), or SPRQPPLLL (SEQ ID NO: 9).

A number of other methods may be used for generating T cells in vitro. For example, autologous tumor-infiltrating lymphocytes can be used in the generation of CTL. Plebanski et al. (Plebanski et al., 1995, which is incorporated by reference in its entirety) made use of autologous peripheral blood lymphocytes (PLBs) in the preparation of T cells. Furthermore, the production of autologous T cells by pulsing dendritic cells with peptide or polypeptide, or via infection with recombinant virus is possible. Also, B cells can be used in the production of autologous T cells. In addition, macrophages pulsed with peptide or polypeptide, or infected with recombinant virus, may be used in the preparation of autologous T cells. S. Walter et al. (Walter et al., 2003, which is incorporated by reference in its entirety) describe the in vitro priming of T cells by using artificial antigen presenting cells (aAPCs), which is also a suitable way for generating T cells against the peptide of choice. In the present disclosure, aAPCs were generated by the coupling of preformed MHC:peptide complexes to the surface of polystyrene particles (microbeads) by biotin:streptavidin biochemistry. This system permits the exact control of the MHC density on aAPCs, which allows to selectively elicit high- or low-avidity antigen-specific T cell responses with high efficiency from blood samples. Apart from MHC:peptide complexes, aAPCs should carry other proteins with co-stimulatory activity like anti-CD28 antibodies coupled to their surface. Furthermore, such aAPC-based systems often require the addition of appropriate soluble factors, e. g. cytokines, like interleukin-12.

Allogeneic cells may also be used in the preparation of T cells and a method is described in detail in WO 97/26328, incorporated herein by reference. For example, in addition to Drosophila cells and T2 cells, other cells may be used to present antigens such as CHO cells, baculovirus-infected insect cells, bacteria, yeast, and vaccinia-infected target cells. In addition, plant viruses may be used (see, for example, Porta et al. (Porta et al., 1994, which is incorporated by reference in its entirety) which describes the development of cowpea mosaic virus as a high-yielding system for the presentation of foreign peptides.

The activated T cells that are directed against the peptides of the disclosure are useful in therapy. Thus, a further aspect of the disclosure provides activated T cells obtainable by the foregoing methods of the disclosure.

In an aspect, activated T cells, which are produced by the above method, will selectively recognize a cell that aberrantly expresses a polypeptide that comprises an amino acid sequence of RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), or SPRQPPLLL (SEQ ID NO: 9).

Preferably, the T cell recognizes the cell by interacting through its TCR with the HLA/peptide-complex (for example, binding). The T cells are useful in a method of killing target cells in a patient whose target cells aberrantly express a polypeptide comprising an amino acid sequence of the disclosure wherein the patient is administered an effective number of the activated T cells. The T cells that are administered to the patient may be derived from the patient and activated as described above (i.e. they are autologous T cells). Alternatively, the T cells are not from the patient but are from another individual. Of course, it is preferred if the individual is a healthy individual. By “healthy individual” it is meant that the individual is generally in good health, preferably has a competent immune system and, more preferably, is not suffering from any disease that can be readily tested for, and detected.

In vivo, the target cells for the CD8-positive T cells according to the present disclosure can be cells of the tumor (which sometimes express MHC class II) and/or stromal cells surrounding the tumor (tumor cells) (which sometimes also express MHC class II; (Dengjel et al., 2006, which is incorporated by reference in its entirety)).

The T cells of the present disclosure may be used as active ingredients of a therapeutic composition. Thus, the disclosure also provides a method of killing target cells in a patient whose target cells aberrantly express a polypeptide comprising an amino acid sequence of the disclosure, the method comprising administering to the patient an effective number of T cells as defined above.

The HLA phenotype, transcriptomic and peptidomic data is gathered from the patient's tumor material, and blood samples to identify the most suitable peptides for each patient containing “warehouse” and patient-unique (i.e. mutated) TUMAPs. Those peptides will be chosen, which are selectively or over-expressed in the patients' tumor and, where possible, show strong in vitro immunogenicity if tested with the patients' individual PBMCs.

### EXAMPLES

### Example 1

Isolation of MHC Epitopes from Tissue Samples

Tissue Samples

This example describes a representative methodology for the isolation of epitopes.

1,514 different healthy and tumour samples were obtained from primary tissues of 1,119 donors. Written informed consents of all individuals had been given before surgery or autopsy. Tissues were shock-frozen immediately after excision and stored until isolation of MHC epitopes at −70° C. or below.

Isolation of HLA epitopes from tissue samples

HLA peptide pools from shock-frozen tissue samples were obtained by immune precipitation from solid tissues according to a slightly modified protocol (Falk et al., 1991; Seeger et al., 1999, which is incorporated by reference in its entirety) using the HLA-A*02-specific antibody 887.2, the HLA-A, -B, -C specific antibody W6/32, CNBr-activated sepharose, acid treatment, and ultrafiltration.

Mass Spectrometry Analyses

The HLA peptide pools as obtained were separated according to their hydrophobicity by reversed-phase chromatography (nanoAcquity UPLC system, Waters) and the eluting peptides were analyzed in L TQ-velos and fusion hybrid mass spectrometers (ThermoElectron) equipped with an ESI source. Peptide pools were loaded directly onto the analytical fused-silica micro-capillary column (75 μm i.d.×250 mm) packed with 1.7 μm C 18 reversed-phase material (Waters) applying a flow rate of 400 nL per minute. Subsequently, the peptides were separated using a two-step 180 minute-binary gradient from 10% to 33% Bat a flow rate of 300 nL per minute. The gradient was composed of Solvent A (0.1% formic acid in water) and solvent B (0.1% formic acid in acetonitrile). A gold coated glass capillary (PicoTip, New Objective) was used for introduction into the nanoESI source. The L TQ-Orbitrap mass spectrometers were operated in the data dependent mode using a TOPS strategy. In brief, a scan cycle was initiated with a full scan of high mass accuracy in the Orbitrap (R=30 000), which was followed by MS/MS scans also in the Orbitrap (R=7500) on the 5 most abundant precursor ions with dynamic exclusion of previously selected ions. Tandem mass spectra were interpreted by SEQUEST and additional manual control. The identified peptide sequence was confirmed by comparison of the generated natural peptide fragmentation pattern with the fragmentation pattern of a synthetic sequence-identical reference peptide.

Label-free relative LC-MS quantitation was performed by ion counting i.e. by extraction and analysis of LC-MS features (Mueller et al., 2007 which is incorporated by reference in its entirety). The method assumes that the peptide's LC-MS signal area correlates with its abundance in the sample. Extracted features were further processed by charge state deconvolution and retention time alignment (Mueller et al., 2008; Sturm et al., 2008, which is incorporated by reference in its entirety). Finally, all LC-MS features were cross-referenced with the sequence identification results to combine quantitative data of different samples and tissues to peptide presentation profiles. The quantitative data were normalized in a two-tier fashion according to central tendency to account for variation within technical and biological replicates. Thus, each identified peptide can be associated with quantitative data allowing relative quantification between samples and tissues. In addition, all quantitative data acquired for peptide candidates was inspected manually to assure data consistency and to verify the accuracy of the automated analysis. For each peptide a presentation profile was calculated showing the mean sample presentation as well as replicate variations. The profiles juxtapose cancer samples to a baseline of normal tissue samples.

### Example 2

In Vitro Immunogenicity of MHC Class I Presented Peptides

To obtain information regarding the immunogenicity of the peptides of the present disclosure, an in vitro T-cell priming assay based on repeated stimulations of CD8+ T cells with artificial antigen presenting cells (aAPCs) loaded with peptide/MHC complexes and anti-CD28 antibody may be performed.

In Vitro Priming of CD8+ T Cells

To perform in vitro stimulations by artificial antigen presenting cells loaded with peptide-MHC complex (pMHC) and anti-CD28 antibody, CD8+ T cells from fresh HLA-A*02 leukapheresis products may be isolated via positive selection using CD8 microbeads (Miltenyi Biotec, Bergisch-Gladbach, Germany) of healthy donors obtained from the University clinics Mannheim, Germany, after informed consent. PBMCs and isolated CD8+ lymphocytes were incubated in T-cell medium (TCM) until use consisting of RPMI-Glutamax (Invitrogen, Karlsruhe, Germany) supplemented with 10% heat inactivated human AB serum (PAN-Biotech, Aidenbach, Germany), 100 U/ml Penicillin/100 μg/ml Streptomycin (Cambrex, Cologne, Germany), 1 mM sodium pyruvate (CC Pro, Oberdorla, Germany), 20 μg/ml Gentamycin (Cambrex). 2.5 ng/ml IL-7 (PromoCell, Heidelberg, Germany) and 10 U/ml IL-2 (Novartis Pharma, Nurnberg, Germany) were also added to the TCM at this step. Generation of pMHC/anti-CD28 coated beads, T-cell stimulations and readout was performed in a highly defined in vitro system using four different pMHC molecules per stimulation condition and 8 different pMHC molecules per readout condition.

The purified co-stimulatory mouse IgG2a anti human CD28 Ab 9.3 (Jung et al., 1987) was chemically biotinylated using Sulfo-N-hydroxysuccinimidobiotin as recommended by the manufacturer (Perbio, Bonn, Germany). Beads used were 5.6 μm diameter streptavidin coated polystyrene particles (Bangs Laboratories, Illinois, USA).

800.000 beads/200 μl may be coated in 96-well plates in the presence of 4×12.5 ng different biotin-pMHC, washed and 600 ng biotin anti-CD28 were added subsequently in a volume of 200 μl. Stimulations may be initiated in 96-well plates by co-incubating 1×10⁶ CD8+ T cells with 2×10⁵ washed coated beads in 200 μl TCM supplemented with 5 ng/ml IL-12 (PromoCell) for 3 days at 37° C. Half of the medium may be then exchanged by fresh TCM supplemented with 80 U/ml IL-2 and incubating may be continued for 4 days at 37° C.

This stimulation cycle may be performed for a total of three times. For the pMHC multimer readout using 8 different pMHC molecules per condition, a two-dimensional combinatorial coding approach may be used as previously described (Andersen et al., 2012, which is incorporated by reference in its entirety) with minor modifications encompassing coupling to 5 different fluorochromes. Finally, multimeric analyses may be performed by staining the cells with Live/dead near IR dye (Invitrogen, Karlsruhe, Germany), CD8-FITCO antibody clone SK1 (BD, Heidelberg, Germany) and fluorescent pMHC multimers. For analysis, a BD LSRII SORP cytometer equipped with appropriate lasers and filters may be used. Peptide specific cells may be calculated as percentage of total CD8+ cells. Evaluation of multimeric analysis may be performed using the FlowJo software (Tree Star, Oregon, USA). In vitro priming of specific multimer+ CD8+ lymphocytes may be detected by comparing to negative control stimulations. Immunogenicity for a given antigen may be detected if at least one evaluable in vitro stimulated well of one healthy donor was found to contain a specific CD8+ T-cell line after in vitro stimulation (i.e. this well contained at least 1% of specific multimer+ among CD8+ T-cells and the percentage of specific multimer+ cells was at least 10× the median of the negative control stimulations).

### Example 3

Synthesis of Peptides

Peptides may be synthesized using standard and established solid phase peptide synthesis using the Fmoc-strategy. Identity and purity of each individual peptide may be determined by mass spectrometry and analytical RP-HPLC. The peptides may be obtained as white to off-white lyophilizates (trifluoro acetate salt) in purities of >50%. Peptides may be administered as trifluoro-acetate salts or acetate salts, other salt-forms are also possible.

### Example 4

Identification of RNA Editing-Derived Epitopes

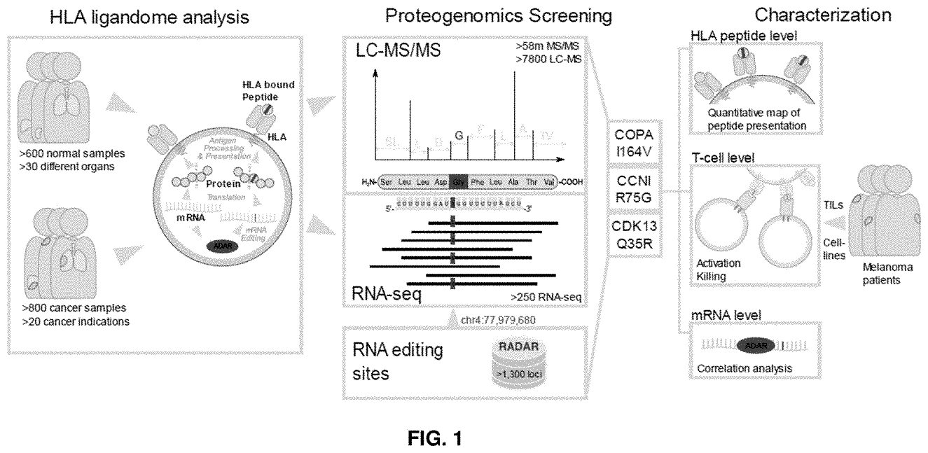

FIG. 1 shows proteogenomics-guided discovery of HLA peptides derived from RNA editing and characterization of edited cyclin I (CCNI) as T cell epitope and personalized cancer target predictable by mRNA biomarker. Pipeline combining RNA-seq and LC-MS data from primary tissue for discovery of HLA ligands derived from RNA editing sites listed in RADAR. CCNI peptides were quantitatively analysed and compiled into an in vivo map of peptide abundance to assess tumour association. In parallel, deeper target characterization by assessment of immunogenicity and T cell killing was performed. For further validation, correlation between peptide and mRNA levels of edited CCNI and ADAR were assessed. To identify HLA-presented peptides derived from RNA editing (ED), data acquired by the antigen discovery platform XPRESIDENT® that combines liquid chromatography-mass spectrometry (LC-MS) for identification and quantitation of HLA ligands with RNA sequencing (RNA-seq) of corresponding mRNA was investigated.

HLA class I-peptides were isolated from 1,514 different healthy and tumour samples from primary tissues of 1,119 donors resulting in approximately 60 million fragment spectra (MS/MS). To search for EDs, the present inventors first constructed an RNA editome peptide database, which contains RNA editome peptide database used in screening for HLA-bound peptides. This database contains a total of 2,516 entries for 1,387 edited peptides and their WT counterparts, which are derived from 1,369 unique RNA editing sites. Each edited site is flanked by 10 amino acids according to the corresponding protein sequence. RNA editome peptide database was derived from 1,369 loci extracted from the Rigorously Annotated Database of A-to-I RNA editing (RADAR).

Table 1 shows that matching MS/MS spectra against the database identified the following peptides, i.e., RVWDVSGLRK (SEQ ID NO: 1), RVWDVSGLRKK (SEQ ID NO: 2), LLDGFLATV (SEQ ID NO: 3), SLLDGFLATV (SEQ ID NO: 4), AENALESYAFN (SEQ ID NO: 5), GLADGVMQCSF (SEQ ID NO: 7), and SPRQPPLLL (SEQ ID NO: 9).

TABLE 1

| RNA-seq | | | | LC-MS evidence | | | |
|---|---|---|---|---|---|---|---|
| | | | | | | | Peptide- |
| Editing | #RNA- | Editing | | | | Donor HLA | spectrum |
| site | seq | level % | FDR % | Peptide ion | #LC-MS | restriction | matching |
| COPA 1164V | 245 | 13.2 | 0.07 | RVWDVESGLRK⁻³ | 13 | A*03:11 | successful |
| | | | 0.07 | RVWDVSGLRKK⁻³ | 6 | A*03:01 | successful |
| CCN1 R75G | 252 | 5.1 | 2.40 | LLDGFLATV⁻² | 46 | A*02:01 | successful |
| | | | 0.56 | SLLDGFLATV⁻¹ | 44 | A*02:01 | successful |
| | | | 0.19 | SLLDGFLATV⁻² | 6 | A*02:01 | successful |
| HSPA1L | 2 | 0 | 0.33 | AENALE3YAFN⁻³ | 2 | B*44:03 | AENAEFMRNF⁻² |
| K541E | | | | | | | |
| FRRS1 R295G | 17 | 0 | 0.43 | GLADGVMQCSF⁻³ | 2 | A*02:06 | AVADDFCKV⁻² |
| | | | | (M7 oxidized) | | | (C7 w/ Cys- |
| | | | | | | | Gly adduct) |
| CDK13 Q35R | 20 | 8.8 | 0.96 | SPRQPPLLL⁻² | 24 | B*07:02 | successful |

| SEQ ID | | | |
|---|---|---|---|
| NO: | Amino Acid Sequence | Peptide Name | Editing Site |
| 1 | RVWDVSGLRK | COPA-ED10 | COPA I164V |
| 2 | RVWDVSGLRKK | COPA-ED11 | COPA I164V |
| 3 | LLDGFLATV | CCNI-ED9 | CCNI R75G |
| 4 | SLLDGFLATV | CCNI-ED10 | CCNI R75G |
| 5 | AENALESYAFN | HSPA1L-1 | HSPA1L K541E |
| 6 | AENAEFMRNF | HSPA1L-2 | HSPA1L K541E |
| 7 | GLADGVMQCSF | FRRS1-1 | FRRS1 R295G |
| 8 | AVADDFCKV | FRRS1-2 | FRRS1 R295G |
| 9 | SPRQPPLLL | CDK13-ED | CDK13 Q35R |

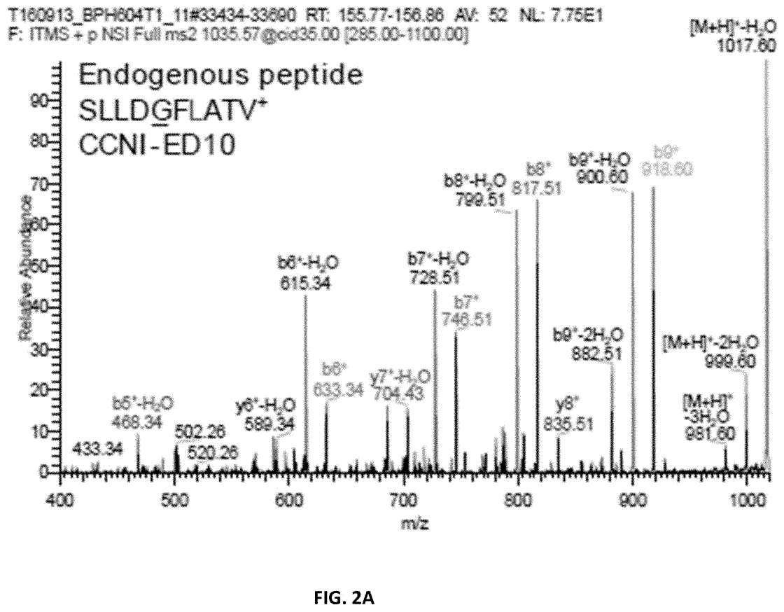

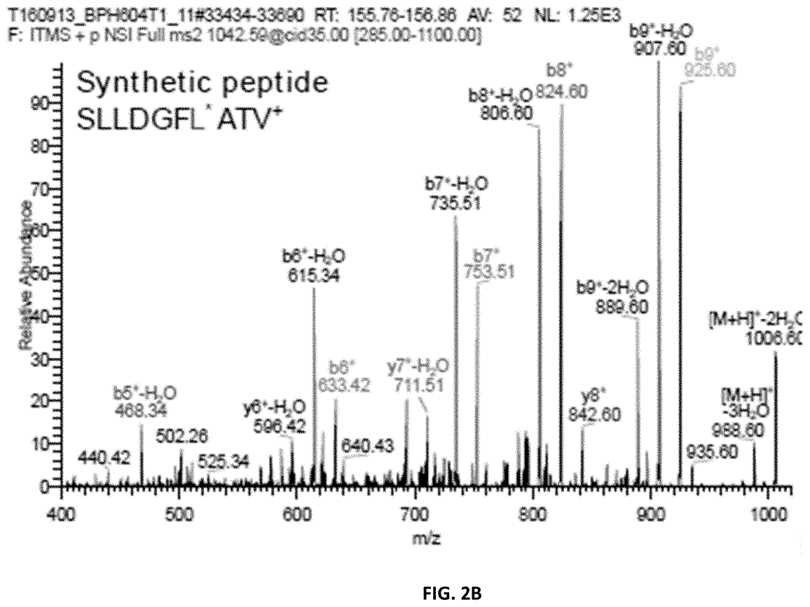

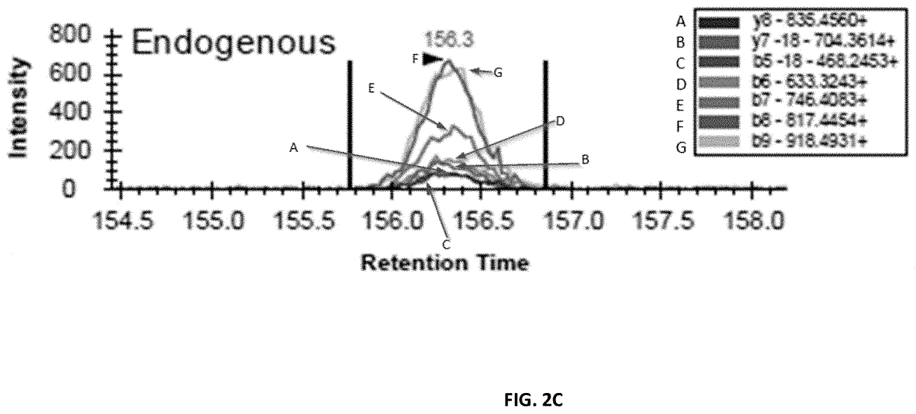

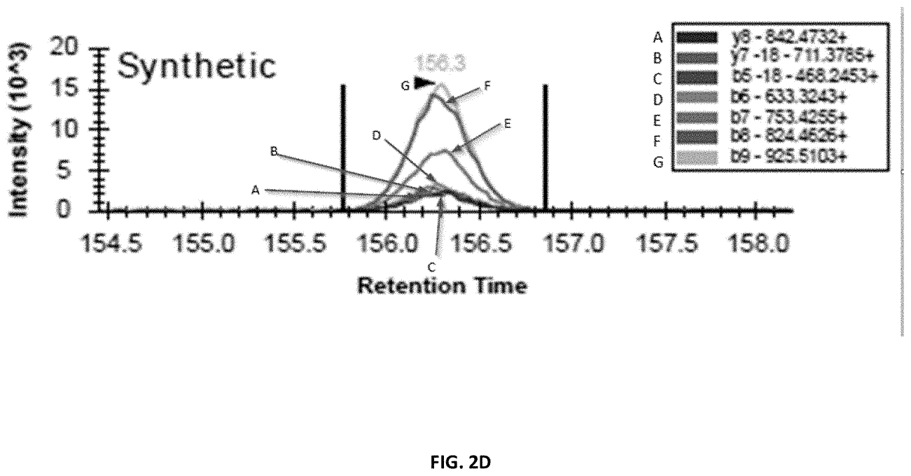

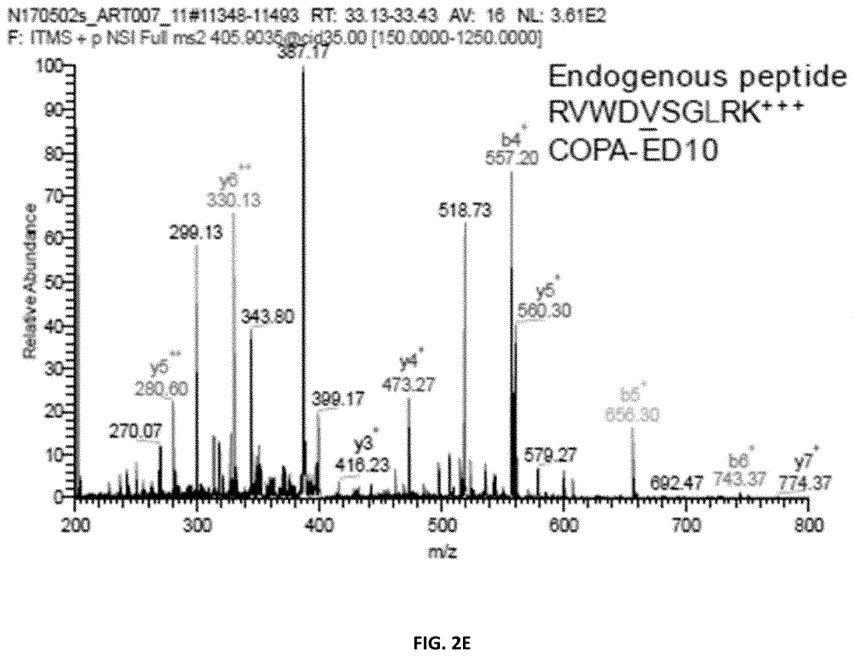

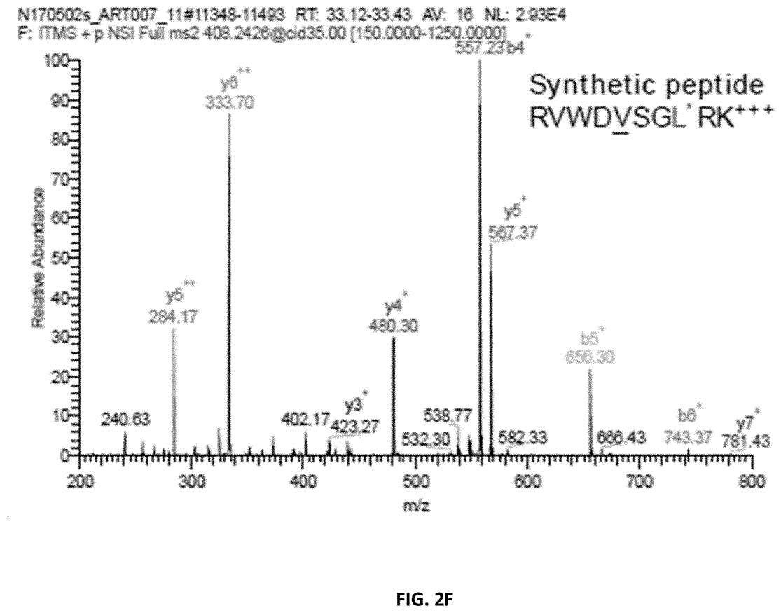

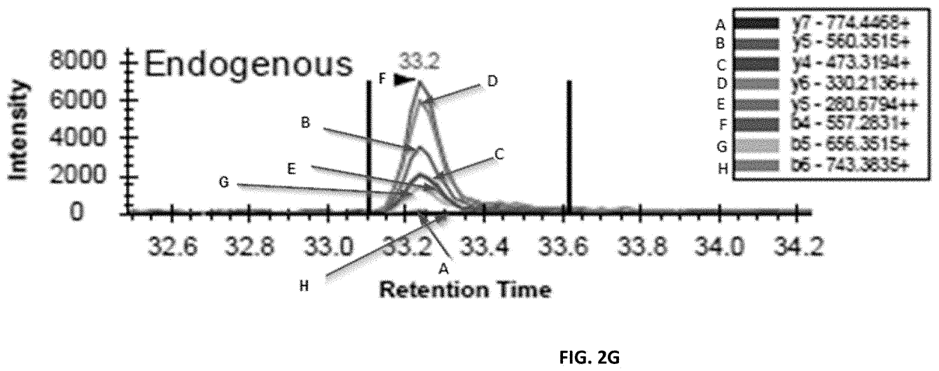

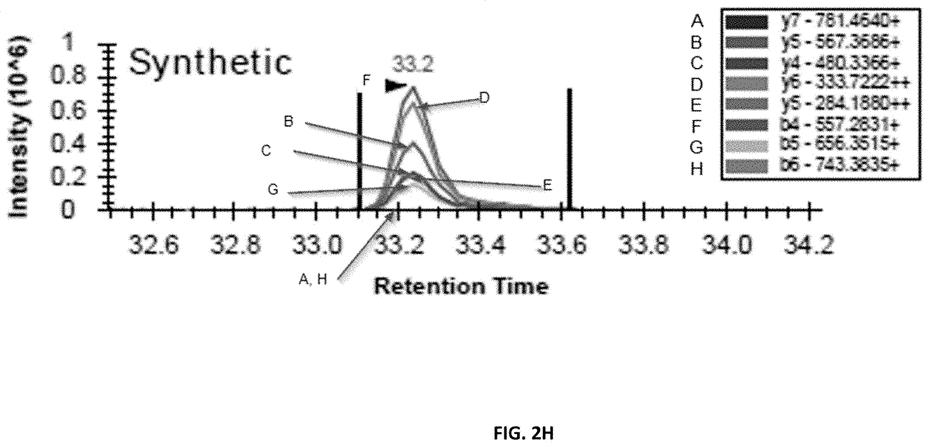

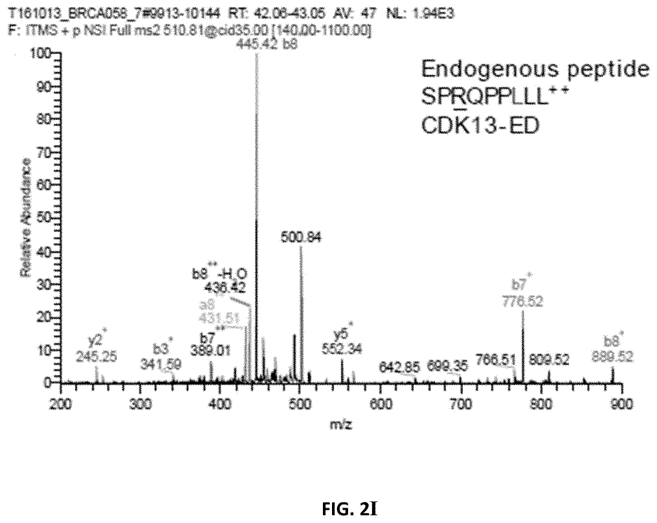

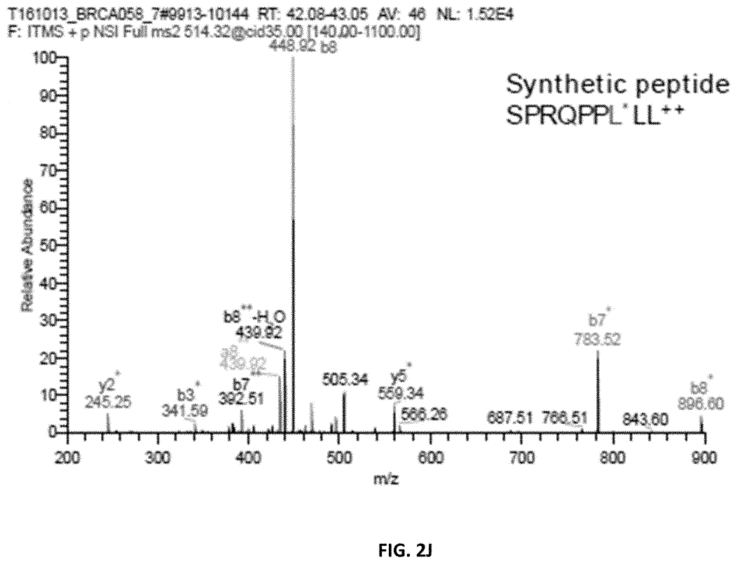

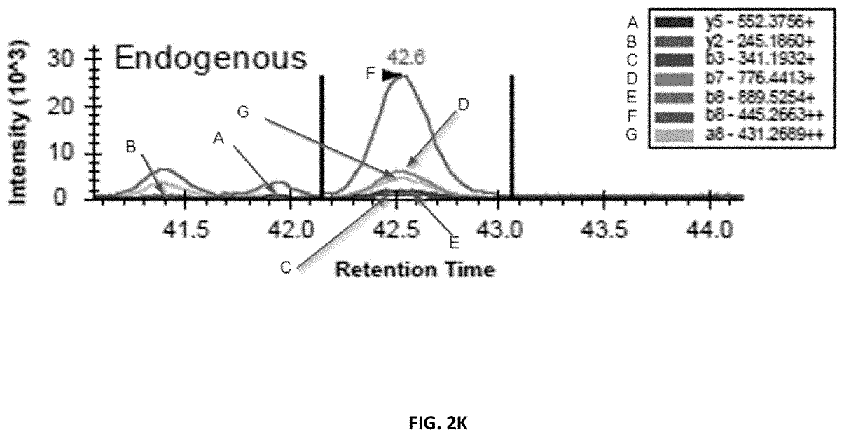

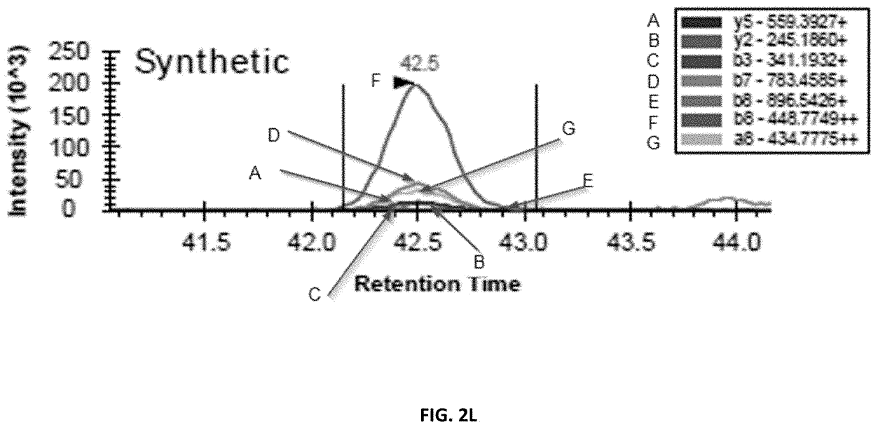

FIGS. 2A and 2C show fragmentation pattern and retention time of endogenous peptides eluted from tumour sample for singly charged endogenous CCNI-ED10 (2A, 2C).

FIGS. 2E and 2G show fragmentation pattern and retention time of endogenous peptides eluted from tumour sample for triply charged COPA-ED10 (2E, 2G).

FIGS. 2I and 2K show fragmentation pattern and retention time of endogenous peptides eluted from tumour sample for doubly charged CDK13-ED.

FIGS. 2B and 2D show matching fragmentation pattern and retention times for CCNI-ED10 and prove sequence identity.

FIGS. 2F and 2H show matching fragmentation pattern and retention times for COPA-ED10 and prove sequence identity.

FIGS. 2J and 2L show matching fragmentation pattern and retention times for and CDK13-ED and prove sequence identity.

Synthetic reference peptides isotopically labelled either at Leucine seven (CCNI-ED10, CDK13-ED) or eight (COPA-ED10) were spiked into the same tumour sample and are expected to coelute due to identical physicochemical properties if the sequences are identical. Fragments carrying the label show a 7 Da mass shift. The peptides, e.g., CCNI-ED10: SLLDGFLATV (SEQ ID NO: 4) (2A-2D), COPA-ED10: RVWDVSGLRK (SEQ ID NO: 1) (2E-2H), and CDK13-ED: SPRQPPLLL (SEQ ID NO: 9) (2I-2L), were confirmed by coelution of corresponding synthetic isotope-labelled peptides using LC-MS.

Table 2 lists the confirmed EDs of which four were derived from the well described editing sites CCNI R75G and COPA I164V as well as CDK13 Q35R previously identified exclusively by RNA-seq. Due to the immunopurification step, there was confirmation that these peptides are HLA ligands whereas DNA-based HLA typing of the corresponding donors allowed to infer the HLA restriction of each peptide. Each of the two peptides found for CCNI and COPA formed nested sets. For each ED, the corresponding non-edited wild type peptide (WT) was also detected. While the number of identified edited peptides seems low, based on RNA-seq, only 160 editing sites of the searched editome were expressed in the samples used for immunopeptidomics. Thus, this proteogenomic gap between the number of RNA events and HLA peptides detected is in the range of other proteogenomics studies on SNPs and somatic mutations.

TABLE 2

| | | HLA | | Peptide |
|---|---|---|---|---|
| Editing site | Gene | restriction | Peptide name | sequence |
| CCNI R75G | Cyclin I | A*02:01 | CCNI-WT9 | LLDRFLATV |
| chr4:77,979,680 | | | CCNI-ED9 | LLDGFLATV |
| | | | CCNI-WT10 | SLLDRFLATV |
| | | | CCNI-ED10 | SLLDGFLATV |
| COPA I164V | Coatomer | A*03:01 | COPA-WT10 | RVWDISGLRK |
| chr1:160,302244 | subunt | | COPA-ED10 | RVWDVSGLRK |
| | alpha | | COPA-WT11 | RVWDISGLRKK |
| | | | COPA-ED11 | RVWDVSGLRKK |
| CDK13 Q35R | Cyclin | B*07:0,2 | CDK13-WT | SPQQPPLLL |
| chr7:39,990,34 | Dependent | | CDK13-ED | SPRQPPLLL |
| | Kinase 13 | | | |

| SEQ ID | Amino Acid | | |
|---|---|---|---|
| NO: | Sequence | Peptide Name | Editing Site |
| 10 | LLDRFLATV | CCNI-WT9 | CCNI R75G |
| 3 | LLDGFLATV | CCNI-ED9 | CCNI R75G |
| 11 | SLLDRFLATV | CCNI-WT10 | CCNI R75G |
| 4 | SLLDGFLATV | CCNI-ED10 | CCNI R75G |
| 12 | RVWDISGLRK | COPA-WT10 | COPA I164V |
| 1 | RVWDVSGLRK | COPA-ED10 | COPA I164V |
| 13 | RVWDISGLRKK | COPA-WT11 | COPA I164V |
| 2 | RVWDVSGLRKK | COPA-ED11 | COPA I164V |
| 14 | SPQQPPLLL | CDK13-WT | CDK13 Q35R |
| 9 | SPRQPPLLL | CDK13-ED | CDK13 Q35R |

Table 2 shows confirmation of HLA class I-bound EDs identified by proteogenomics screening. Three editing sites were identified and for each information about the gene and the amino acid substitution (chromosomal coordinates in GRCh37/hg19) are shown. The associated edited peptides (ED) with editing site underlined and their non-edited wildtype counterparts (WT) are listed. HLA restrictions were determined based on HLA typing of corresponding DNA and specificity of the immunoprecipitation antibody.

Abundances of RNA Editing-Derived Epitopes in Tumors

Since RNA editing was reported to be associated with cancer, the HLA-A*02 ligand CCNI-ED were selected for in-depth characterization making use of quantitative HLA-A*02 peptidome data for 925 samples. This helped assess the tumour association of CCNI peptides in a comprehensive and unbiased fashion by directly analysing their presentation levels on HLA in cancer (n=504) and normal tissues (n=421).

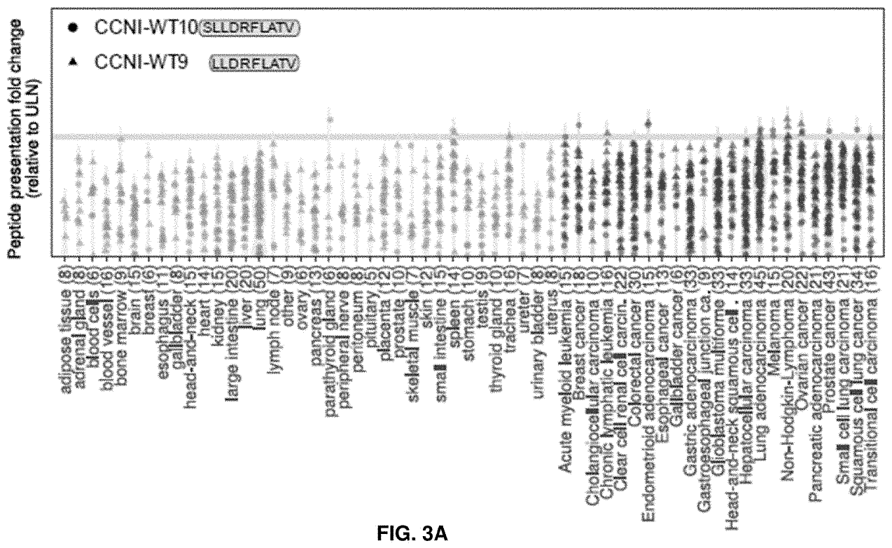

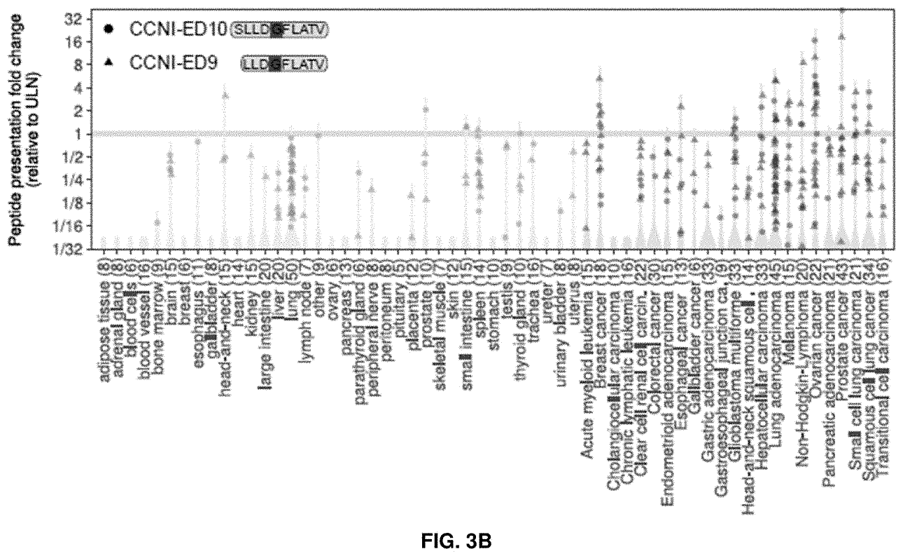

FIGS. 3A and 3B show relative abundance of HLA-bound peptides derived from non-edited wild type (WT) and edited (ED) CCNI peptides isolated from tumour (red) and normal samples (blue), respectively. Each dot represents a sample for which the peptide was detected. Samples are grouped according to healthy organ or tumour indication. Total number of donors per group is indicated in parentheses. LC-MS signals were expressed as fold change relative to the upper limit of normal (ULN, grey line). Violin plots are superimposed to visualize the distribution of all samples including those with low (< 1/32 ULN) or without peptide detection. FIG. 3A shows both CCNI-WT peptides were detected in almost all A*02 positive samples, showing similar levels in normal and cancer tissues, while CCNI-ED showed elevated abundances in several tumors (FIG. 3B).

To identify samples with unusually high editing, the healthy sample population was used to define a normal reference range and the upper limit of normal (ULN) in particular. Samples with HLA peptide abundance above ULN were defined as hyper-edited. Of the 504 tumors quantified, 36 (7%) showed hyper-editing of up to 40-fold above ULN. The observed prevalence is comparable to the most frequent neoantigen epitopes like PIK3CA H1047R, which is expressed in 4% of all tumours. Considering individual tumor indications, the most prevalent indications are ovarian cancer (41%, n=9/22), melanoma (27%, n=4/15) and breast cancer (22%, n=4/18). Again, this is comparable to frequent neoantigen epitopes likes KRAS G12D showing a prevalence in pancreatic cancer of 33%.

Stimulation of T Cell Response by RNA Editing-Derived Epitopes

To examine whether the detected HLA ligands are recognized by tumor-infiltrating lymphocytes (TILs) and if these TILs can kill cells presenting ED. For this analysis, matched autologous pairs of melanoma TILs and tumor cell lines were used. CCNI-ED peptide and its WT counterpart were synthesized and evaluated for their ability to activate TILs generated from human melanoma tumours.

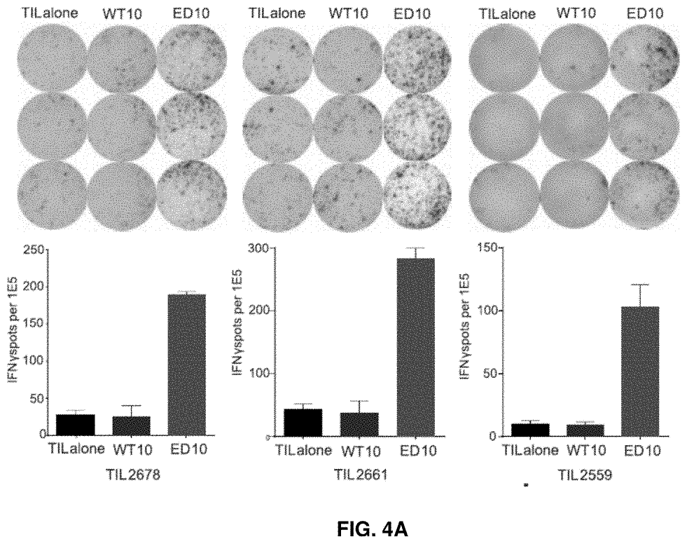

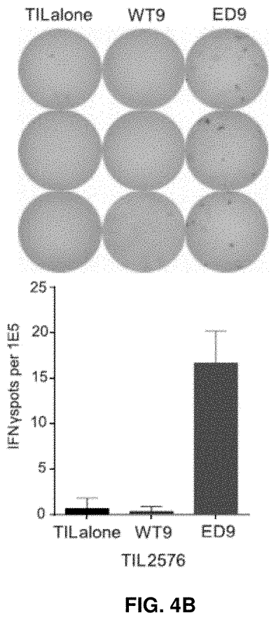

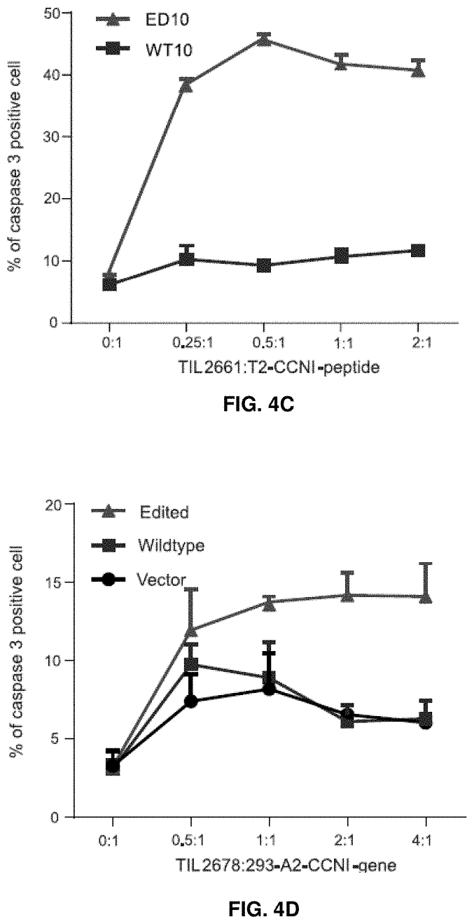

FIG. 4A shows ELISPOT (upper) and summary graphs (lower) showing IFNγ production (arithmetic mean and SEM, n=3) by 3 melanoma TILs following incubation with CCNI-ED10 peptide. Only background level of IFNγ-producing TILs was detected when incubated alone or together with CCNI-WT10. This result shows 3 out of 15 of HLA-A*02:01-expressing TILs (TIL2678, TIL2661 and TIL2559) strongly responded to CCNI-ED10, as demonstrated by robust production of the T-cell effector cytokine IFNγ in ELISPOT assays run in triplicates, whereas none of the TILs responded to CCNI-WT10. This result suggests the existence of effector T cells in tumor-infiltrating immune cells specifically reacting to CCNI-ED10 implying its in vivo function as antigenic epitope.

FIG. 4B shows a parallel assay using the same TILs identified one that reacted to CCNI-ED9, albeit more weakly than the T-cell responses to CCNI-ED10. Thus, these data demonstrate that edited peptides can function as antigens to stimulate T-cell responses in tumor tissue. This result shows TIL2576 weakly reacted to CCNI-ED9.

T cells play a primary role in adaptive immunity against infections and tumorigenesis, and therapies based on manipulating T-cell activation have shown promise in cancer treatment. To determine the function of CCNI-ED specific T cells in mediating tumor cell killing, the HLA-A*02:01 expressing lymphoblast cell line T2, which is lacking expression of the transporter associated with antigen processing (TAP) and thus incapable of presenting endogenous peptides, were employed. The CCNI-WT or CCNI-ED peptide were pulsed onto T2 cells, co-cultured with TIL2661 in different ratios, and then measured T-cell mediated target cell death based on anti-caspase3 staining and subsequent flow cytometric quantification.

FIG. 4C shows caspase3 based Cytotoxic T Lymphocyte (CTL) killing assay showing TIL2661 mediated killing of T2 cells pulsed with CCNI-ED10 or CCNI-WT. This result shows a pulse of CCNI-ED10, rather than CCNI-WT10, to T2 cells facilitated target killing, suggesting that CCNI-ED10 was a target of cytotoxic T lymphocyte (CTL) activity.

To confirm whether CCNI-ED could also mediate target killing activity under natural antigen processing conditions, the cDNA encoded wildtype or edited CCNI full-length protein were cloned and transiently transfected the HLA-A*02:01-expressing human embryonic kidney cell line (HEK 293-A2).

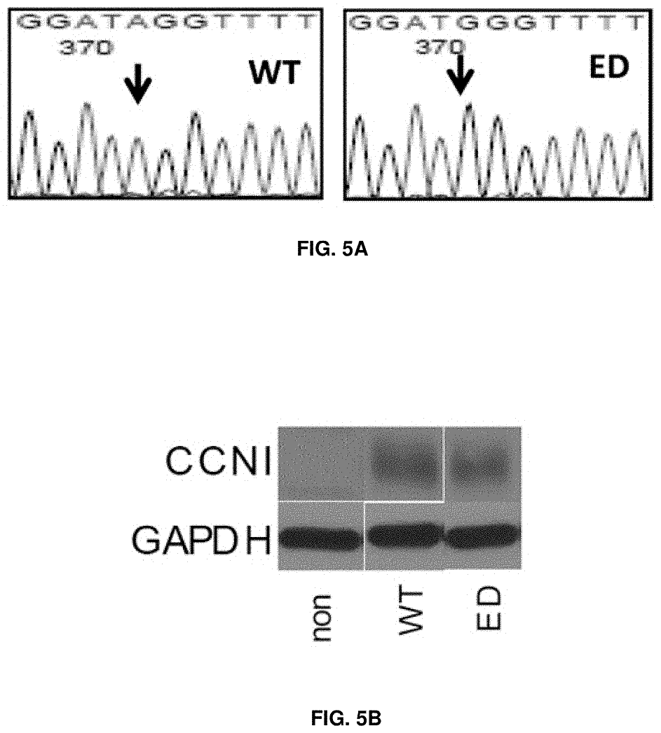

FIG. 5A shows CCNI R75G editing in the DNA construct was confirmed by PCR and sequencing. The arrow indicating the nucleotide AGG in wildtype was changed to GGG in edited cDNA. FIG. 5B shows CCNI protein overexpression in transfected 293-A2 cells were confirmed by immunoblotting blotting. These results confirm CCNI wildtype and edited gene and protein expression. CCNI wildtype and edited genes were cloned into pHAGE vector by Gateway cloning system and transiently transfected into 293-A2 cells. After transfection, total RNA was isolated and RT-PCR was performed to amplify CCNI mRNA using the primers that flanked the CCNI R75G editing site. This was followed by PCR product purification and sequencing to confirm wildtype and edited cDNA expression.

FIG. 4D shows over-expression of edited but not wildtype CCNI gene in 293-A2 cells enhanced TIL2678's CTL killing activity. The error bar represents the standard error of the mean (SEM) of the three replicates. This result shows over-expression of edited protein in 293-A2 was associated with profound cytotoxic activity of TIL2678, whereas expression of wildtype cDNA or empty vector resulted in background levels of cytotoxic activity.

Correlation Between mRNA Editing Level and Number of RNA Editing-Derived Epitopes

The data described above demonstrated CCNI-ED10 as a HLA ligand, that is hyper-edited in a subpopulation of tumor samples, and able to function as a T-cell epitope to stimulate CTL activity. To examine whether CCNI-ED10 can be used as a tumor antigen for personalized immunotherapies, which would require a companion diagnostic for patient selection, the method of choice are mRNA-based measurements assuming that edited mRNA levels indeed correlate with the number of edited peptides bound to HLA. While an overall correlation between transcript levels and number of detectable HLA peptides has been shown, the peptide-specific abundance levels correlate only for a fraction of HLA peptides with their corresponding mRNA. Making use of the quantitative HLA peptidomics data shown in FIGS. 3A-3B, the peptidome data were integrated with corresponding RNA-seq measurements of matched samples to correlate the peptide abundance with the expression of edited CCNI. The result revealed a moderate correlation for CCNI-ED9 (R=0.33, 95% confidence interval CI=0.03-057), but a strong correlation for CCNI-ED10 (R=0.67, CI=0.45-0.81). Taking both length variants into account to determine overall editing on peptide level was accomplished by absolute quantitation measuring the number of edited peptide copies per cell. A set of 8 samples was successfully analyzed by parallel reaction monitoring (PRM) LC-MS and RNA-seq targeting CCNI peptides and corresponding mRNA, respectively.

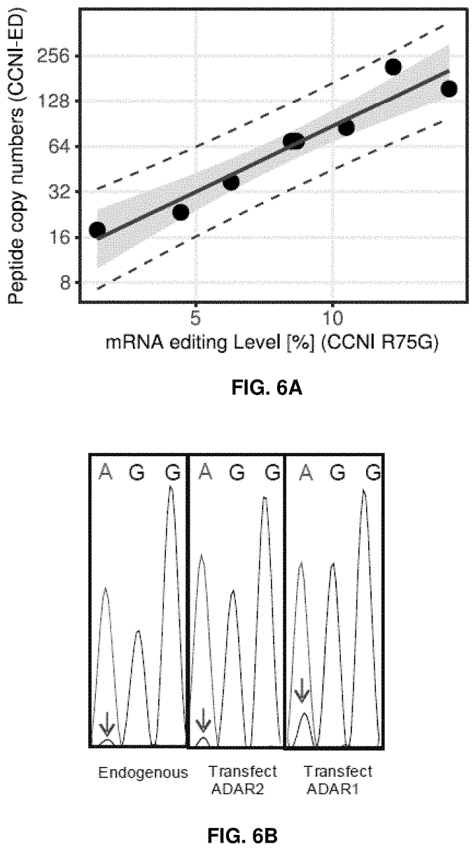

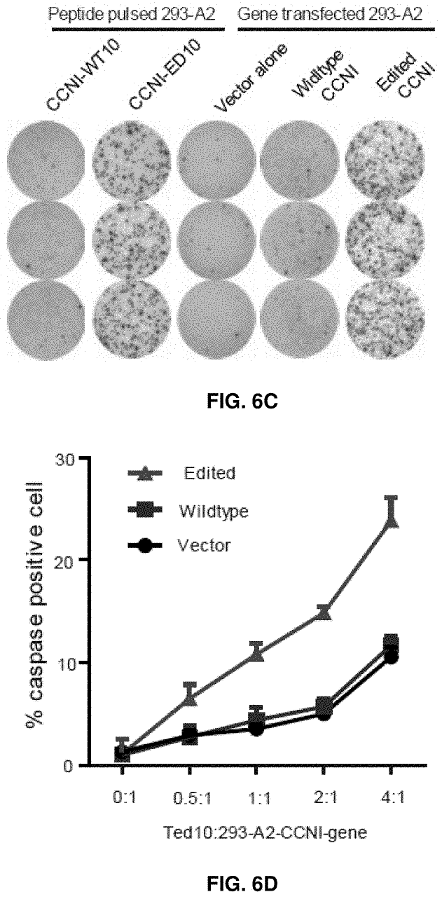

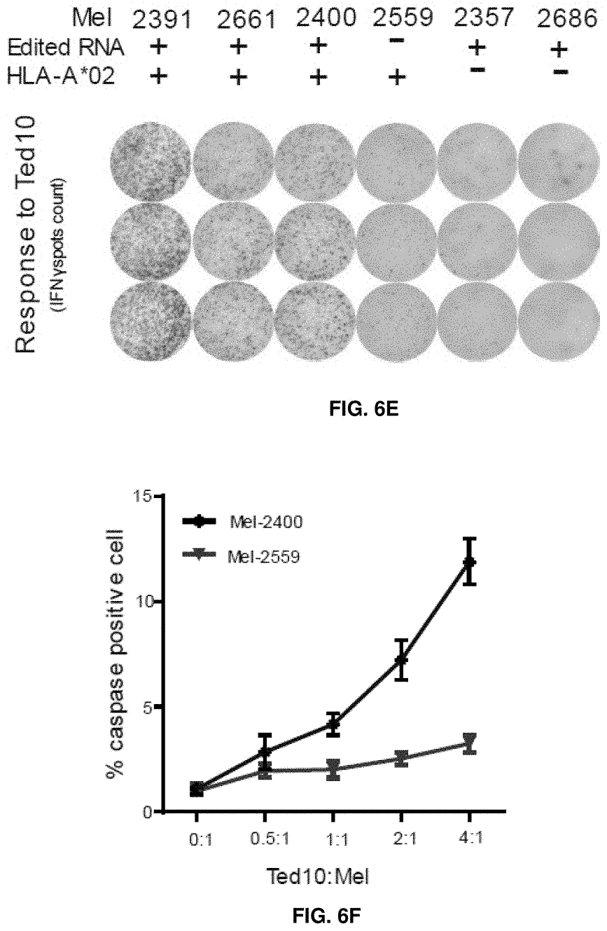

FIG. 6A shows correlation between mRNA editing level and edited peptide copy numbers in accordance with one embodiment of the present disclosure. This result shows correlation between the number of edited CCNI peptide copies per cell determined by the AbsQuant™ method and mRNA editing levels determined by targeted RNA-seq for CCNI R75G. The scatterplot (n=8) includes the regression curve (red line) as well as the 95% confidence interval (grey band) and 95% prediction interval (dashed lines). This result shows high correlation (R=0.96, CI=0.80-0.99) between mRNA editing level and edited peptide copy numbers. Based on this correlation, the prevalence of hyper-editing on mRNA level in The Cancer Genome Atlas (TCGA) for external validation on a larger cohort was investigated.

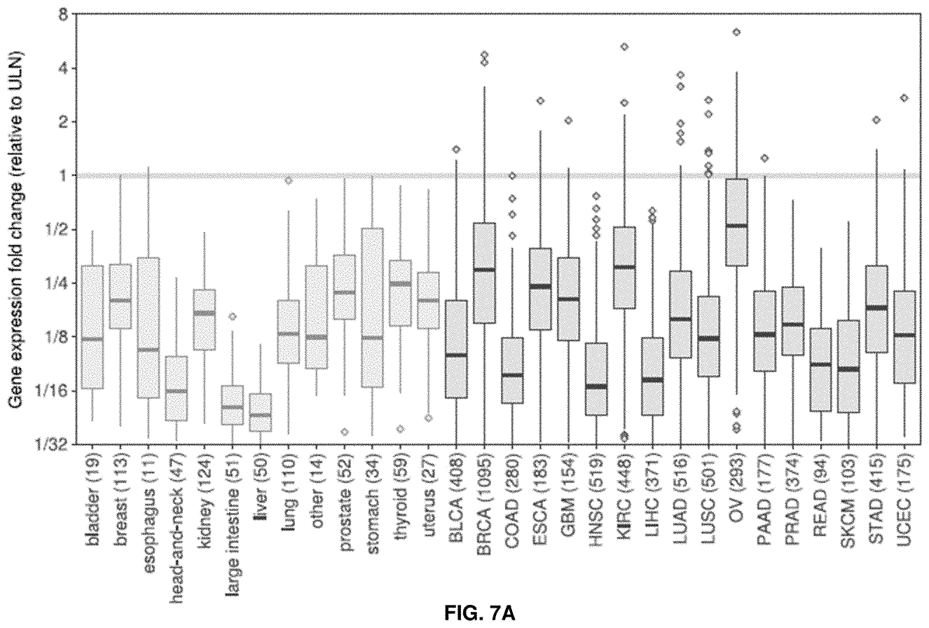

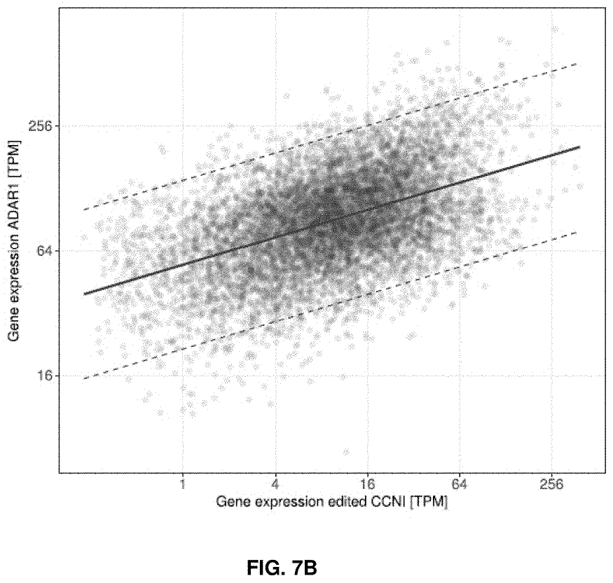

FIG. 7A shows gene expression of edited CCNI mRNA in TCGA tumours against normal tissues. The number of samples per group is given in parentheses. Normalized read counts were expressed as fold change relative to the upper limit of normal (grey line). The distribution of fold changes is displayed as box plot scaled by sample size and highlighting outliers as diamonds. Hyper-editing analysis was performed in the same fashion as on peptide level by determining the ULN and the fold change of edited transcript relative to the ULN. For the TCGA studies matching the cancer types investigated on HLA peptidome level, the results show a prevalence of hyper-editing of 3.4% (n=210/6106) with the highest prevalence for ovarian cancer (OV, 23%, n=67/293), breast cancer (BRCA, 8%, 87/1095) and kidney cancer (KIRC, 6%, n=26/448).

Correlation Between ADAR1 Expression and Editing Gene Expression

Having established the correlation between edited peptide and mRNA, TCGA tumor data were used to extend mechanistic understanding of CCNI editing. There are three members in the ADAR family, of which ADAR1 and ADAR2 have been shown to play major roles in RNA editing.

FIG. 7B shows correlation of gene expression between ADAR1 and edited CCNI for TCGA tumours (R=0.48, n=8,241). The scatterplot includes the regression curve (red line) as well as the 95% prediction interval (dashed lines). This result shows the expression of ADAR transcripts correlated with expression of edited CCNI. The observation that higher correlation for ADAR1 (R=0.48, CI=0.47-0.50) than for ADAR2 (R=0.28, CI=0.26-0.30) or ADAR3 (R=0.07, CI=0.05-0.10) suggests ADAR1 as most likely enzyme responsible for catalyzing the editing. This is also supported by associating the quantitative HLA-A*02 peptidome data (FIG. 1) directly with the corresponding mRNA expression measurements. Logistic regression modelling of the detection of CCNI-ED10 by ADAR1 mRNA expression showed significant association for ADAR1 (Odds ratio OR=30.8, p<0.001) in contrast to ADAR2 (OR=2.1, p=0.157) and ADAR3 (OR=0.8, p=0.572).

To provide additional experimental evidence, HEK 293 were transfected with ADAR1 and ADAR2. FIG. 6B shows CCNI-R75G is edited by ADAR1. HEK 293 stably expressing CCNI wildtype gene was transfected with empty vector or expression vectors of ADAR1 or ADAR2. CCNI editing was measured by RT-PCR and followed by sequencing. The double peaks indicate nucleotide A to G conversion and the height of peaks reflect the level of editing. This result shows elevated CCNI editing only for ADAR1, supporting a relationship.

To investigate this relationship on cellular level, CCNI-ED10-specific effector T cells (Ted10) were generated from HLA-A*02:01-expressing normal peripheral blood mononuclear cells (PBMCs) using an established method. FIG. 6C shows ELISPOT assay. The result shows IFNγ production by Ted10 incubated with peptide-pulsed or CCNI-transfected 293-A2 cells. Ted10 cells were potently activated by 293-A2 cells pulsed with CCNI-ED10 or transfected with the edited gene. Furthermore, FIG. 6D shows CTL killing assay. The result shows over-expression of edited CCNI gene increases the sensitivity of Ted10 mediated 293-A2 target killing (n=3), summarized as mean±SEM per titration. Thus, Ted10 cells displayed substantially elevated killing activity towards 293-A2 cells over-expressing the edited gene compared to background killing activity towards control cells transfected with the wildtype or empty vector.

Endogenous RNA Editing-Derived Epitopes in Tumors

Activation of TIL2661 and TIL2559 (FIG. 4A) by CCNI-ED10 suggests that this epitope is presented endogenously by autologous tumours. Based on the established correlation between edited mRNA and peptide, RNA-sequencing analyses were performed to detect CCNI mRNA editing in melanoma cell lines, including mel-2661, mel-2559 and mel-2400 derived from the patients used for generating TIL2661 and TIL2559.

To examine the functional significance of endogenous CCNI-ED10 in T-cell activation using Ted10, IFNγ ELISPOT assays were used and detected strong reactivity of Ted10 to mel-2400 and mel-2661 as well as another CCNI editing-positive tumor line, mel-2391, whereas Ted10 cells displayed only a background level response to the CCNI-editing negative mel-2559. Furthermore, Ted10 did not react to the HLA-A*02:01 negative mel-2686 and mel-2357 despite their expression of edited mRNA, confirming HLA-restricted and antigen-specific T-cell activation. For example, FIG. 6E shows IFNγ ELISPOT assay, in which recognition of endogenous CCNI-ED antigen by Ted10. Mel-2391, mel-2400 and mel-2661 expressing both edited CCNI mRNA and HLA-A*02:01 are highly reactive to Ted10. Mel-2559, which was derived from the same patient as mel-2400 but does not have detectible edited CCNI mRNA, only reacted at background levels to Ted10. Mel-2357 and mel-2686, which express edited CCNI mRNA but do not express HLA-A*02:01, have no response to Ted10. This result shows endogenously edited CCNI mRNA were detected except for mel-2559, which was derived from the same patient as mel-2400 yet from a different tumor sample. ADAR1 mRNA was 40 times lower in mel-2559 (ΔCt=0.073) compared to mel-2400 (ΔCt=2.93) based on qPCR measurements.

FIG. 6F shows Ted10 mediated target killing following incubation with mel-2400 and mel-2559 measured by caspase-3-based CTL killing assay (summarized as mean±SEM of the three triplicates). This result shows, in parallel CTL assays, Ted10 displayed strong killing activity towards the CCNI-editing positive mel-2400 but had almost no activity against the autologous CCNI-editing negative mel-2559.

ADAR1 Knockdown Reduces T Cell Stimulation by RNA Editing-Derived Epitopes

To knock down ADAR1 mRNA, Mel-2400 cells were stably transduced with Lentivirus that was made of either empty Lentiviral vector (GFP) control (Sh control) or shRNA ADAR1 knock-down construct (Sh ADAR1). Cells were sorted based on GFP expression to isolate transduction positive cells.

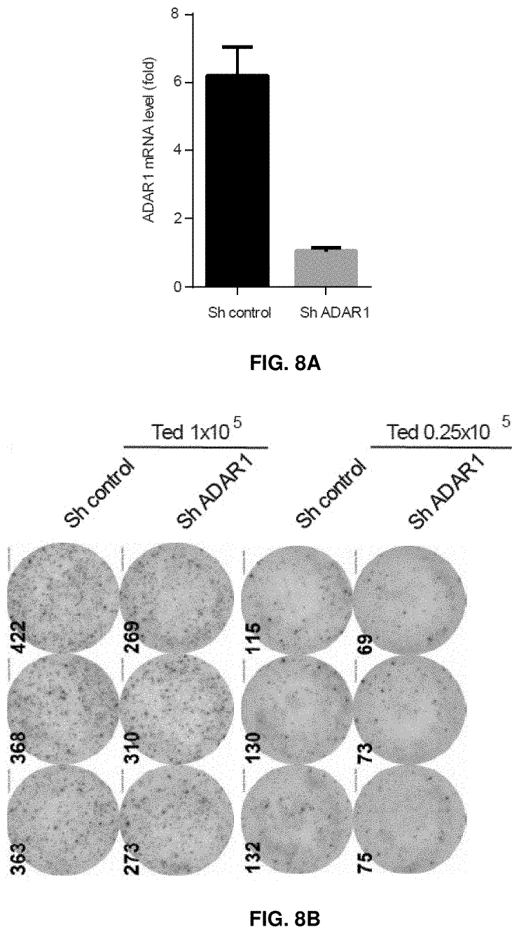

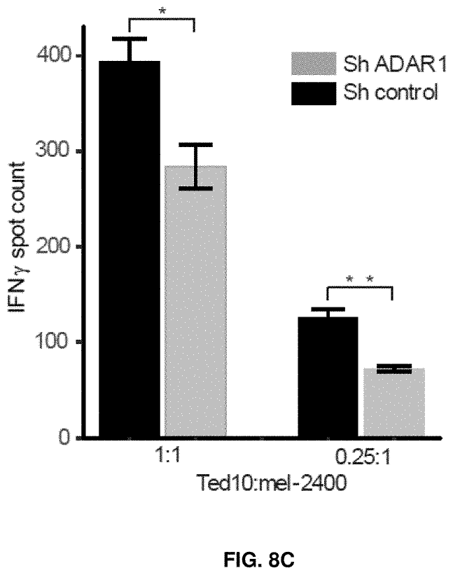

FIG. 8A shows ADAR1 mRNA was greatly reduced in Sh ADAR1 expressing mel-2400 compared with the control cells determined by quantitative RT-PCR and normalized to housekeeping gene GAPDH. Mean fold change and standard error of the mean (SEM) is shown for three independent PCR reactions. The result shows that ADAR1 mRNA levels are significantly reduced in Mel-2400 cells transduced with shRNA ADAR1 knock-down construct than that with the control construct.

FIG. 8B shows ELISPOT assay. The result shows reduced response of Ted10 to mel-2400 cells after knockdown of ADAR1. 1×10⁵ ShADAR1 stably expressed mel-2400 cells or control cells were incubated with 1×10⁵ of Ted10 (left two columns) or 0.25×10⁵ of Ted10 (right two columns) in triplicate for 18 hours and activated Ted10 cells were measured by ELISPOT assay to detect IFNγ production. Knockdown of ADAR1 in mel2400 greatly reduced its ability to stimulate Ted10 cell to produce IFNγ. For 1:1 ratio of Ted10 to mel-2400, the mean spot number is 384:284 (P=0.01200). For 0.5:1 ratio of Ted10 to mel2400, the mean spots number is 126:72 (P=0.024). The data was analyzed by unpaired T test. FIG. 8C shows summary graphs of FIG. 8B. For each condition arithmetic mean±SEM (n=3) is reported. These results show knockdown of ADAR1 in mel-2400 greatly reduced its ability to stimulate Ted10 to produce IFNγ, i.e., down regulation of ADAR1 reduces response of mel-2400 to Ted10 T cells. Thus, ELISPOT assay shows that knockdown of ADAR1 mRNA in CCNI-ED10 positive mel-2400 reduced its ability to activate Ted10 cells to produce IFNγ.

By way of the disclosure, identified RNA editing products, for example, the CCNI-ED10 peptide, were identified as immunogenic epitopes that are able to stimulate T-cell responses. The epitope's target potential was characterized by MS-based immunopeptidomics showing a quantitative profile of an RNA edited HLA-bound peptide on a comprehensive panel of primary human A*02+ tissues as well as direct correlation between peptide level and mRNA. Both synthetic and endogenously expressed CCNI-ED10 peptide could serve as antigen for CTL activation and render tumor cells as efficient killing targets. ED10-specific T cells were detected from both TILs and normal PBMCs, highlighting the in vivo relevance of this edited antigen in eliciting immune responses. These results provide compelling evidence that RNA editing can generate antigenic epitopes, which in turn suggest new opportunities for immunotherapies in the treatment of cancer and immunological disorders.

Methods

Peptide Isolation and Mass Spectrometry

To allow discovery and selection of novel HLA peptides as targets for immunotherapy, the immunopeptidomes were acquired together with the corresponding transcriptomes and HLA genotypes for 1,514 primary human tissue samples extracted post mortem or surgically from 850 patients with cancer or benign neoplasms and 269 healthy tissue donors after written informed consent. The resulting sample set of 616 normal and 898 cancer samples covered 35 different organs and 23 tumor types with at least 5 donors per group and a median group size of 16 donors. Samples were snap-frozen in liquid nitrogen and stored until isolation at −80° C. After tissue homogenization and lysis, peptide-MHC complexes were isolated by immunoprecipitation using class I specific antibodies coupled to CNBr-activated Sepharose resin (GE Healthcare Europe, Freiburg, Germany). Depending on the donor's HLA type the following antibodies were used as previously described24: w6/32 for pan-class I, BB7.2 for HLA-A*02, GAP-A3 for HLA-A*03 and B1.23.2 for HLA-B/C (Department of Immunology, University of Tübingen, Germany). Peptides were eluted from antibody-resin by acid treatment and purified by ultrafiltration. For further separation by reversed-phase chromatography (nanoAcquity UPLC system, Waters, Milford, Mass.) an analytical fused-silica micro-capillary column (75 μm internal diameter×250 mm) was used packed with 1.7 μm C18 and a gradient composed of Solvent A (0.1% formic acid in water) and Solvent B (0.1% formic acid in acetonitrile) that went from 10% to 30% B at a flow rate of 300 nL per minute. Eluted peptides were analyzed by data-dependent acquisition (DDA) in an Orbitrap mass spectrometer (Thermo Fisher Scientific, Waltham, Mass.) equipped with a nano electrospray ionization (ESI) source. A total of 7,825 runs was acquired in profile mode covering most samples with five replicate injections making use of different mass analyzers in low- (TOP3, ion trap acquiring top 3 precursors) and high-resolution mode (TOPS, Orbitrap acquiring top 5 precursors, R=7,500) as well as different fragmentations using collision-induced dissociation (CID) and higher-energy collisional dissociation (HCD). Survey scans were acquired with high mass accuracy in the Orbitrap (R=30,000 for TOP3, R=60,000 for TOPS). Mass range for selection of doubly charged precursors was 400-750 m/z and 800-1500 m/z for singly charged precursors. Spectra were extracted and centroided using Proteome Discoverer 1.4 (Thermo Fisher Scientific, Waltham, Mass.).

RNA Isolation and Sequencing

Immunopeptidome measurements were accompanied by paired transcriptome analysis for a subset of 276 samples by isolating total RNA using TRIzol® (Invitrogen, Karlsruhe, Germany) followed by a purification with the RNeasy mini kit (QIAGEN, Hilden, Germany) according to the manufacturer's protocol. The actual RNA sequencing and expression quantification was performed by CeGaT (Tübingen, Germany). Briefly, 1-2 μg total RNA were used as starting material for the library preparation performed according to the IIlumina® protocol (TruSeq Stranded mRNA Library Prep Kit). The sequencing process was performed on an IIlumina® HiSeq® 2500 machine. For all experiments, a strand-specific protocol was used to generate single-end reads of a length of 50 nucleotides. The minimum number of reads was 43,700,000 per sample. The quality of the sequencing process was monitored using PhiX spike-ins. DESeq25 was performed to determine normalization factors to allow inter-sample read count comparisons.

For eight samples with detectable copy numbers of edited peptide and remaining mRNA available, expression of edited CCNI was measured by CeGaT (Tübingen, Germany) using targeted RNA-seq. Briefly, 100 ng total RNA were used and amplified specifically for CCNI R75G using the primers 5′-GATGTGGAAAGTGAATGTGCG-3′ (forward) (SEQ ID NO: 15) and 5′-TTTGGATGAGCCTTTACGGTAG-3′ (reverse) (SEQ ID NO: 16). The library preparation was performed according to IIlumina® protocol (Nextera XT Index PCR System) followed by sequencing on an HiSeq® 2500 generating about 10 million paired-end reads with length of 2×100 nucleotides.

HLA Typing

To experimentally assess the HLA restriction of a given peptide, DNA of every donor was isolated from tissue or whole blood using the QIAamp® DNA Mini Kit (Qiagen) or the QIAamp® DNA Blood Mini Kit (Qiagen), respectively. The QIAamp® Investigator Kit (Qiagen) has been used to isolate DNA from very limited amounts of tissue. HLA genotyping for HLA-A*02 was performed by PCR and subsequent agarose gel electrophoresis using the Ambisolv® Primer Mix PM002 (Life Technologies) and recombinant Taq polymerase (Life Technologies). Fine typing of the HLA-A and -B loci were performed by Sanger sequencing using the SeCore® Sequencing Kits (Invitrogen/Life Technologies). SeCore® Custom GSSP Kits (Invitrogen/Life Technologies) were used to resolve ambiguities if necessary. Samples were sequenced on an ABI-3100 sequencer (Applied Biosystems/Thermo Fisher Scientific) at CeGaT (Tübingen, Germany) and results were evaluated using the uTYPE™ software (Invitrogen/Life Technologies).

Peptide Identification

RNA editing sites were downloaded from the RADAR version 2 (rnaedit.com) containing 2,576,459 entries. RNA editing sites were annotated using ANNOVAR based on the RefSeq annotations hg19_refGeneMrna.fa and hg19_refGene.txt (www.openbioinformatics.org/annovar/). Filtering for non-synonymous events resulted in 1,369 RNA editing sites. Amino acid sequences were inferred using the R package sapFinder (bioconductor.org/packages/sapFinder). Due to different protein isoforms, the editing sites correspond to 1,387 different candidate peptides with up to ten amino acids before and after the editing site. The editing peptide database was concatenated with the reference proteome (UniProt 2016 Apr. 13, www.uniprot.org) and a reversed version thereof for MS/MS database search using Comet (v2016.01 rev.2, comet-ms.sourceforge.net/). The search was performed with the following parameters: peptide length 8-12 AAs, mass range 700-1500 Da, non-specific enzymatic digestion, precursor mass tolerance 3 ppm, 0.02 Da bin size for high resolution (Orbitrap) spectra and 1 Da for low resolution (Ion trap) spectra, and methionine oxidation as variable modification. The Comet search results were then analyzed by PeptideProphet (TPP v5.0.0, tools.proteomecenter.org) which estimates a probability score for each Peptide-spectrum-match (PSM) with assistance of decoy hit scores. All the PSMs from all samples were further grouped into individual peptide ions (distinct peptide sequence, modification, and charge state) and the best probability score was taken to represent the final score for each peptide ion. False discovery rate (FDR) for each peptide ion was then estimated by target-decoy approach. Table 1 lists all RNA editing sites found at 1% FDR as well as all peptide ions at 5% FDR that cover those sites including different length variants and charge states. The identified peptide ions were inspected for MS/MS matching quality, HLA restriction, and RNA-seq support. MS/MS matching quality was assessed by inspecting the matched fragment ion coverage and coverage of dominant peaks. In case of questionable coverage, alternative sequences suggested by de novo identification using PepNovo+ (2010 Nov. 17, proteomics.ucsd.edu/Software/PepNovo/) were considered. HLA restriction of an identified edited peptide was determined by comparing the potential MHC peptide binding motif with the experimental HLA typing of the corresponding sample and the specificity of the antibody used for immunoprecipitation (see Table 1). RNA-seq experiments of mRNA extracted from the same sample as the peptide eluates were analyzed using samtools 0.1.19 (www.htslib.org) to find supporting reads.

Peptide Sequence Validation

To experimentally validate the edited peptide sequence, peptides were synthesized on an automated Prelude® peptide synthesizer (Protein Technologies Inc., Tucson, Ariz.) using solid phase peptide synthesis (SPPS) and Fmoc-chemistry. C13/N15-labelled Leucine (Cambridge Isotope Laboratories Inc., Tewksbury, Mass.) was used to isotopically label the peptide resulting in a mass shift of 7.017 Da. The isotope-labelled peptides were spiked into retention vials of the original sample and analyzed by LC-MS. The heavy labelled peptide variant creates a reference signal that has the same chromatographic properties yet is not interfering with the native mass signal. To unambiguously validate the sequence identity of edited peptides, the elution times and the fragmentation pattern of labelled and native peptide signals were compared (FIGS. 2A-2L). To detect the peptides with maximum sensitivity, the MS/MS spectra were acquired by data independent mode (DIA) restricting to labelled and native peptide masses. Fragmentation patterns were generated using XCalibur 3.0.63 (Thermo Fisher Scientific) and elution profiles were extracted using Skyline 3.6.0, which is hereby incorporated by reference in its entirety (proteome.gs.washington.edu/software/skyline).

Relative Quantitation of Peptides

For direct quantitation of CCNI peptides on peptide presentation level, 421 normal and 504 tumor samples were chosen based on the following criteria. The donor was tested positive for A*02 based on HLA typing and the BB7.2 immunoprecipitation resulted in at least 100 identified peptides based on at least four evaluable technical replicates. LC-MS peptide signal features were extracted by SuperHirn v1.028 allowing determination of peak areas for extracted ion chromatograms (XIC) allowing MS1-based relative quantitation. After charge state deconvolution with OpenMS Decharger 1.6 (www.openms.de), LC-MS features were assigned to identified MS/MS spectra. To allow maximum sensitivity, identification was based on a spectral library approach using 5% posterior error probability (PEP) fitted by R mixtools 1.1.0 (CRAN.R-project.org/package=mixtools) on cross-correlation scores between manually confirmed reference spectra and all available MS/MS within a 10 ppm precursor range. Peptide abundance levels per sample were determined by median total-area of the replicates. The total-area was defined as the sum of the normalized XIC areas of all observed charge states. Systematic bias was rectified by central tendency normalization to account for differences in MHC expression and technical variations.

Statistical Analysis of Immunopeptidome Data

Statistics and figures were generated using R 3.4.2 (www.r-project.org) and ggplot2 2.2.1 (ggplot2.org). Tumor association of CCNI peptides was analyzed by comparison against the normal reference range. Peptide abundances of normal samples were grouped according to organ and the 95th percentile (P95) was determined for each organ. The upper limit of normal (ULN) was defined as maximum P95 over all healthy organs and tumor samples above ULN were classified as hyper-edited. For visualization, peptide abundances were presented as fold change with respect to ULN and grouped according to tumor type or healthy organ. Every group consisted of at least 5 samples. Normal samples from cartilage, bile duct, eye, thymus, central nerve, spinal cord and pleura did not meet this requirement and were grouped into the category other. The distribution of fold changes for each group was estimated as violin plots. Samples with values below 1/32 ULN or without detection of the peptide were set to 1/32 ULN.

Association between label-free LC-MS and corresponding RNA-seq data was analyzed between CCNI peptides and CCNI as well as ADAR gene expression. To investigate if mRNA levels are predictive for peptide presentation levels, the correlation between peptide quantitation and normalized read count for CCNI R75G edited reads was analyzed by Pearson's correlation coefficient based on all samples with pairwise complete observations (nED9=44, nED10=39). Association with ADAR was analyzed with logistic regression modelling for all pairwise measurements. The likelihood of peptide detection was modelled by log-transformed reads per kilobase per million mapped reads (RPKM) for ADAR1-3.

Absolute Quantitation of Peptides

For a set of 22 samples absolute copy numbers per cell where measured using the AbsQuant™ method. In brief, the copy numbers of CCNI peptides were calculated using the number of cells within the investigated tissue and the total amount of the isolated peptide. Hereby, both parameters were determined experimentally. The isolation efficiency was assumed to be 100%. For three samples determination of cell count was not possible whereas one sample had no mRNA left for performing targeted RNA-seq analysis. Ten samples were not evaluable due to LC-MS issues in detection of one or both CCNI-ED peptides. This resulted in a set of eight samples with evaluable copy numbers.

The number of cells was determined based on the quantitation of DNA content in the investigated human tissue sample. Therefore, DNA was isolated using QIAamp® DNA Mini Kit (QIAGEN, Hilden, Germany) from lysate aliquot which was sampled during the isolation of HLA ligands from primary tissue. The DNA yield was quantified using Qubit™ dsDNA HS Assay Kit (Applied Biosystems/Thermo Fisher Scientific) and the number of cells was interpolated from DNA content using a standard curve derived from peripheral blood mononuclear cells (PBMCs).

For absolute quantitation of CCNI peptides, a series of nanoLC-MS/MS measurements was performed on an Orbitrap mass spectrometer (Thermo Fisher Scientific, Waltham, Mass.) using parallel reaction monitoring (PRM). Two differently isotopically labelled CCNI equivalents were synthesized as described above. One of the isotopically labelled equivalents was used as an absolute quantity reference and was spiked into retention vials of each human tissue sample which was used for absolute quantitation of CCNI peptides. The other isotopically labelled equivalent was used to generate the peptide-specific standard curve. Thereby, one of the isotopically labelled equivalents was titrated and the other one was used as mentioned before as an absolute quantity reference. The MS/MS spectra were acquired by data independent mode (DIA) restricting to labelled peptide masses by the analysis of standard curves and labelled and native peptide masses by the analysis of primary tissue samples. The MS/MS signals of selected fragment ions were extracted using Skyline 3.6.0 and interpolated in absolute peptide amount using peptide-specific standard curves. The number of edited copies per cell was defined as sum of copies for CCNI-ED9 and CCNI-ED10. Values below limit of detection (LOD) or lower limit of quantitation (LLOQ) were imputed with the respective thresholds.

TCGA Data Analysis

9,233 RNA-seq barn files of normal and tumor TCGA samples were downloaded from Cancer Genomics hub (CGhub; cghub.ucsc.edu). The number of edited and total reads at chr4:77,979,680 was extracted as well as gene expression of CCNI and ADAR1 to ADAR3 as transcripts per kilobase million (TPM). Pearson's correlation coefficients between CCNI and ADAR were determined after log-transformation for all tumor samples (n=8,522) with pairwise complete non-zero values (n=8,241 for ADAR1/2, n=6,666 for ADAR3). Hyper-editing analysis was done analogously to the peptide data. For estimation of the reference range, autologous normal samples from TCGA were grouped according to organ if at least 5 samples existed. Normal samples from brain, pancreas, skin, thymus and soft tissue did not meet this requirement and were grouped into the category other. Tumor samples were restricted to studies with patient populations comparable to those used for immunopeptidome quantitation covering bladder urothelial carcinoma (BLCA), glioblastoma multiforme (GBM), hepatocellular carcinoma (LIHC), ovarian serous cystadenocarcinoma (OV), acute myeloid leukaemia (LAML), oesophageal carcinoma (ESCA), stomach adenocarcinoma (STAD), cholangiocarcinoma (CHOL), colon adenocarcinoma (COAD), rectum adenocarcinoma (READ), pancreatic adenocarcinoma (PAAD), lung adenocarcinoma (LUAD), kidney renal clear cell carcinoma (KIRC), lung squamous cell carcinoma (LUSC), prostate adenocarcinoma (PRAD), lymphoid neoplasm diffuse large B-cell lymphoma (DLBC), skin cutaneous melanoma (SKCM), uterine corpus endometrial carcinoma (UCEC), head and neck squamous cell carcinoma (HNSC) and breast invasive carcinoma (BRCA).

Tumor Infiltrating Lymphocytes (TILs) and Tumor Cell Lines Generation

The TILs and tumor cell lines used for experimental validation were derived from leftover tumor tissue obtained from metastatic melanoma patients enrolled on an adoptive cell therapy clinical trial using TILs at the University of Texas MD Anderson Cancer Center (Institutional review board (IRB)-approved protocol #2004-0069, NCT00338377). All patients had granted a written informed consent.

Melanoma TILs were generated as described before. Briefly, melanoma tumor samples were either cut into 1-3 mm² fragments and put in culture in a tissue-treated 24-well plate, in complete TIL media (TIL-CM) consisting of RPMI 1640 (Gibco, 61871), 10% of human AB serum (GEMINI, 100-512), 0.1% of 2-mercaptoethanol (Gibco, 21985023), 1% of HEPES (Corning, 25-060), 1% sodium pyruvate (Invitrogen/Life Technologies, 11360-070), 1% of Glutomax (Gibco, 35050061), 1% PenStrep (ThermoFisher, 15070063) plus 6000 U/mL of human IL-2 or put in culture after the tumor samples were enzymatically digested by collagenase for 1 h at 37° C. followed by centrifugation using a multi-layer Ficoll gradient (100% and 75%, Ficoll-Paque PLUS, GE, 17-1440-02) where the 100% layer was collected for TILs. Every 3 days, half of the medium was replaced with fresh medium and the TIL culture was split to keep the cells at a density of 1×106/mL. TILs were expanded between 2 to 5 weeks, depending on the TIL lines. To increase the number of TILs available for experiments, the lines were further expanded using the rapid expansion protocol (REP). In brief, 1.5×10⁵ primary TILs generated above were cultured with 27×106 feeder cells together with 0.6 mg soluble anti-CD3 monoclonal antibody (OKT3 clone, Muronomab—Abbott Labs). The feeder cells are peripheral blood mononuclear (PBMC) cells mixed from at least 5 healthy donors and irradiated at 5,000 cGy for 20 minutes prior to culture in order to prevent their proliferation during the REP. 6,000 U/mL IL-2 was added at the second day and half of the medium was recovered and replaced with fresh medium containing 50% of TIL-CM and 50% of AIM-V medium (Invitrogen, 12055-083) every 3 days to keep TIL density between 0.5-2×10⁶/mL. The cultured TILs were harvested at day 14 for functional analysis or frozen in human serum with 10% DMSO (Thermo Fisher, 67-68-5). Autologous tumor cell lines were also established from the enzymatically digested tumours followed by the multi-layer Ficoll gradient where the 75% layer was collected and put in culture in complete tumor media (RPMI 1640, Gibco, 61871) containing 10% FBS, 1% HEPES (Corning, 25-060), 1% sodium pyruvate (Invitrogen/Life Technologies, 11360-070), 1% insulin/selenium/transferrin (Gibco, 51300), 0.2% MycoZap-PR (Lonza, VZA-2011). All tumor samples were HLA typed at the HLA-A locus in the MD Anderson HLA Typing Laboratory. The cell lines were routinely tested for mycoplasma contamination (Lonza, LT07-418).

ELISPOT Assay

IFNγ Enzyme-linked immunospot (ELISPOT) assay was performed to detect T-cell responses. MultiScreen 96 well filter plates (Millipore, MAHAS4510) were coated over night at 4° C. with 75 μL/well of 5 ng/mL anti-human IFNγ capture antibody (Mabtech AB, 3420-3-1000). TILs or CCNI-ED specific T cells (Ted10) were thawed and cultured with 1000U of human IL-2/mL overnight. On the next day, before performing ELISPOT assay, T cells were starved with IL-2 free medium for 6 hours. T cells were then added into the plates in triplicates at 2×10⁵ cells/well (for TIL) or 0.4×10⁵/well (for Ted10) or as indicated in each experiment with culture medium either alone or supplemented with peptides (10 μM final concentration), peptide-pulsed T2 (1×10⁵/well), 293-A2 cells (1×10⁵/well) or melanoma cell lines (1×10⁵/well). After 18 hours of cultivation at 37° C. and 5% CO2, the plates were incubated with 1 ng/mL of Biotinylated anti-human IFNγ monoclonal antibody (Mabtech, 3420-6-1000) for one hour, stained with ExtrAvidine®-Alkaline phosphatase (Sigma-Aldrich, E2636) and IFNγ positive spots were detected with BCIP/NBT Membrane Alkaline Phosphatase Substrate (Sigma, 11697471001). Plates were scanned and counted using the ImmunoSpot® ELISPOT analyzer (Shaker Heights, Ohio) to determine the number of spots/well.

Peptides and Tetramers

The synthetic peptides used in this study were obtained from Genemed Synthesis, Inc (San Antonio, Tex.) or were synthesized as described above (Immatics®, TObingen, Germany). All peptides were purified by HPLC to get a homogeneity of >95%. The tetramer was made by Protein Chemistry Core-MHC Tetramer Lab in Baylor College of Medicine (Houston Tex.).

Peptides Pulsing

5×10⁶ T2 or 293-A2 cells in 1 mL of PBS supplemented with 1% FBS (foetal bovine serum) were incubated with synthetic WT or ED peptides at 10 μM of final concentration for 2 hours at 37° C. incubator and washed once with T-cell medium before being subjected to ELISPOT or Caspase-3-based CTL killing assay.

In Vitro Generation of Peptide-Specific T Cells

Peptide-specific T cells were generated from normal donor's peripheral blood mononuclear cells (PBMCs), and leukapheresis were purchased from Key Biologics (Memphis, Tenn.). The adherent monocytes from PBMC were cultured for one week with 800 U/mL of recombinant human GM-CSF (Thermo Fisher, 215-GM) and 500 U/mL of recombinant human IL-4 (R and D, 204-IL-050) to generate dendritic cells (DCs) and then treated for 24 hours with 10 ng/mL of recombinant human TNFα (R and D, 210-TA), 2 ng/mL of recombinant human IL-1β (R and D, 201-LB-005), and 1000 U/mL of recombinant human IL-6 (R and D. 206-IL-010) plus 1000 ng/mL of Prostagladin E2 (MP Biomedicals, 219457601) to induce DC maturation. Usually, 1.8×10⁶ matured DCs were then pulsed with 10 μM peptides for 4 hours at room temperature in PBS supplemented with 1% human serum. Peptides pulsed DCs were then irradiated for 20 minutes at 5000 rad. Autologous PBMC was then mixed with DCs at 35:1 ratio and cultured in T-cell medium supplemented with 30 ng/mL of recombinant human IL-7 (R and D, 1 207-IL-005) and 5 ng/mL of recombinant human IL-21 (PeproTech, AF-200-21) to enhance peptide specific T-cell growth. Two days later, IL-2 (10 U/mL) was added. Every two days, half medium was replaced with fresh medium containing IL-2. After one week, the cultured T cells were stimulated again with DCs as described above. After a total of 3 weeks, CD8 and peptide-tetramer double-positive T cells were stained with PB conjugated anti-CD8 antibody (BD Biosciences Pharmingen, 558207) and PE-conjugated tetramer (Protein Chemistry Core-MHC Tetramer Lab in Baylor College of Medicine) and then sorted at MD Anderson Flow core facility. Sorted T cells were rested in medium overnight and expanded using a 14-day Rapid Expansion (REP) Protocol. After expansion, the peptide-specific T cells were further characterized by flow cytometric analysis based on CD8 and tetramer staining.

Caspase-3 Cleavage Based Cytotoxic T Lymphocyte (CTL) Killing Assay

T cell-mediated cell killing was analyzed using a flow cytometry-based method by detecting T cell-induced caspase-3 cleavage in target cells. The CCNI ED10 peptide-reacting TIL2661, TIL2559, TIL2678 or Ted10 cells were thawed and cultured with 1000 U/mL of IL-2 for overnight. 5×10⁶ of target cells (T2, 293-A2 or melanoma cell lines) were labelled with CellTrace™ far red dye, DDAO-SE (Molecular Probes, C34553) at a final concentration of 0.6 μM for 15 minutes at 37° C. in 1 mL of PBS supplemented with 1% human serum. 5×10⁴ DDAO-labelled target cells then were incubated in triplicates with different ratios of T cells for 3 hours in 96 well plates. T cell-mediated caspase-3 cleavage was measured by intracellular staining with Cytofix/Cytoperm reagent (BD Biosciences, 554772) and PE conjugated anti-cleaved caspase-3 antibody (BD Bioscience, 550821) and the number of pre-apoptotic cells were determined by flow cytometry.

cDNA Constructs

cDNAs for both wildtype and edited CCNI were cloned using the Gateway cloning system. Donor plasmids containing human WT CCNI cDNAs were purchased from Invitrogen. Site-directed mutagenesis (Clontech, 630703) was performed to get the edited cDNA and then cloned into a lentiviral vector, pHAGE (Addgene, 24526) by LR recombination (Thermo Fisher, 11791). All cDNA clones are verified by sequencing at the MD Anderson DNA core facility.

Cell Transfection

1×10⁶ 293-A2 cells were seeded in each well of 6-well tissue culture plate in DMEM medium with 10% FBS to give 80% confluent on the day of transfection. For each well of cells, 2-4 μg of cDNA per 6 well and 8 μL of Lipofectamine 2000 (Invitrogen/Life Technologies, 11668-027) were used following manufacturers' instructions.

ADAR1 Knock Down by Small Hairpin RNA (shRNA)

pSIH-H1-GFP empty vector and pSIH-H1-GFP-ShADAR1 DNA were purchased from System Biosciences. To knock down ADAR1 in melanoma cell lines, a Lentivirus was generated. 8×10⁶ 293 cells were seeded in 100 mM plate until 80% of confluence. The 2nd generation of lentiviral packaging plasmid pCMVR8.74 (Addgene, 22036) and PMD2G envelope expressing plasmid (Addgene, 12259) were co-transfected with pSIH-H1-GFP empty vector or pSIH-H1-GFP-ShADAR1 DNA as describe above. The supernatant containing the virus was harvested at day 2 and day 3 after transfection. Melanoma cell lines were then transduced with filtered viral supernatant plus 10 μg/mL of polybrene (EMD Millipore Corp, TR-1003). Stably transduced cells were then selected based on expression of green fluorescent protein (GFP) after 4 days of transduction.

Protein Analysis by Immunoblotting

293-A2 cells transfected with the indicated lentiviral expression vectors were lysed in RIPA cell lysis buffer (Thermo Fisher scientific, 89900), and cell lysates (1 μg/sample) were subjected to SDS-PAGE and transferred onto nitrocellulose blot membranes for immunoblotting using anti-CCNI antibody (1:2000 dilution, Sigma-Aldrich, GW22274). The same membrane was then striped and re-blotted with an antibody for the housekeeping protein glyceraldehyde 3-phosphate dehydrogenase (GAPDH) as loading control (Santa Cruz sc-32233).

RT-PCR and PCR Product Sequencing

Total RNA was isolated using Qiagen mini RNeasy kit (Qiagen 74104) and subjected to cDNA synthesis using a high-capacity cDNA kit (Thermo Fisher scientific, 4368813). The primers that flank the editing site of CCNI mRNA were used to amplify CCNI DNA fragment. The CCNI PCR primers used were forward primer: 5′-CACTAGGGAAGCACAGATGTG-3′ (SEQ ID NO: 17) and reverse primer: 5′-CCAATGGTGTGGCTGTGTGAAG-3′ (SEQ ID NO: 18). PCR product was then purified using Qiagen QIAquick PCR Purification Kit (Qiagen, 28104). The purified PCR product was sequenced at the DNA sequencing core facility at MD Anderson.

Quantitative PCR (qPCR)

Total RNA was isolated and converted to cDNA as described above. qPCR was performed using iTaq™ Universal SYBR® Green Supermix reagent (Bio Red, 1725122) in a C1000™ Thermal Cycler CFX96 Real-Time System following manufacturer's instructions (Bio-Rad). The primers used for amplification of ADAR1 were 5′-GACACCGRCACTGCCACCTTC-3′ (forward) (SEQ ID NO: 19) and 5′-GGTAGATACTCAGTTCCTGG-3′ (reverse) (SEQ ID NO: 20). The house-keeping gene GAPDH was used for normalization and amplified with 5′-CATCATCTCTGCCCCCTCT-3′ (forward) (SEQ ID NO: 21) and 5′-GGTGCTAAGCAGTTGGTGGT-3′ (reverse) (SEQ ID NO: 22).

RNA Sequencing of Melanoma Cell Lines

To analyze CCNI and other RNA editing event in melanoma cell lines, RNA sequencing analysis was performed. RNA was isolated from melanoma cell lines using RNeasy mini kit (Qiagen) and subjected to next generation RNA sequencing at the Deep Sequencing Core Facility of MDACC.

Data Availability

The list of screened RNA editing sites is available as supplementary table. HLA ligandomics LC-MS/MS data supporting peptide sequence identifications can be downloaded at www.peptideatlas.org/PASS/PASS01150.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior disclosure.

1. A method for identifying an RNA editing-derived epitope, comprising
isolating a plurality of MHC epitopes from an individual,
selecting, from the plurality of MHC epitopes, an MHC epitope comprising an edited amino acid sequence,
activating a MHC epitope-specific T cell in peripheral blood mononuclear cells (PBMC) by contacting the PBMC with an antigen presenting cell presenting the selected MHC epitope on the cell surface,
isolating the activated MHC epitope-specific T cell from the PBMC,
contacting the isolated activated MHC epitope-specific T cell with a target cell, and
identifying the selected MHC epitope as the RNA editing-derived epitope.

2. The method of claim 1, wherein the edited amino acid sequence is obtained from an RNA editome peptide database containing an RNA editing site and the corresponding amino acid sequence.

3. The method of claim 1, further comprising performing a mass spectrometry analysis on the plurality of MHC epitopes to generate a high-resolution spectra and a low-resolution spectra for each of the plurality of MHC epitopes.

4. The method of claim 3, wherein the selected MHC epitope has a length of from 8 to 12 amino acids and mass spectrometer parameters comprising a mass range about 700-1500 Da, a precursor mass tolerance about 3 ppm, about 0.02 Da bin size for the high resolution spectra, and about 1 Da for the low resolution spectra.

5. (canceled)

6. (canceled)

7. The method of claim 1, wherein the MHC epitope is identified as an RNA editing-derived epitope if contacting the isolated activated MHC epitope-specific T cell with the target cell elicits an immune response against the target cell.

8. The method of claim 1, wherein the antigen presenting cell is a dendritic cell.

9. The method of claim 1, wherein the RNA editing-derived epitope elicits an immune response in an individual and optionally wherein the immune response comprises a cytotoxic T cell response.

10. The method of claim 9, wherein the immune response comprises IFN-γ release by the isolated activated MHC epitope-specific T cell.

11.-13. (canceled)

14. The method of claim 1, wherein the individual expresses an RNA-specific adenosine deaminase (ADAR) gene.

15. The method of claim 14, wherein the ADAR gene is ADAR1 gene.

16. The method of claim 14, wherein the ADAR converts adenosine to inosine.

17. The method of claim 1, wherein the target cell presents the RNA editing-derived epitope in a complex with a MHC molecule on the cell surface.

18.-60. (canceled)

61. A method for identifying an RNA editing-derived epitope, comprising
isolating a plurality of MHC epitopes from an individual,
selecting, from the plurality of MHC epitopes, an MHC epitope comprising an edited amino acid sequence,
activating a MHC epitope-specific T cell,
isolating the activated MHC epitope-specific T cell,
contacting the isolated activated MHC epitope-specific T cell with a target cell, and
identifying the selected MHC epitope as the RNA editing-derived epitope.

62.-85. (canceled)
